(19) United States
(12) Patent Application Publication
(10) Pub. No.: US 2003/0039658 A1
(43) Pub. Date: Feb. 27, 2003

(54) MCEF, a novel transcription factor

(76) Inventors: Mario Estable, Toronto (CA); Robert A. Roeder, New York, NY (US)

Correspondence Address: KLAUBER & JACKSON, 411 HACKENSACK AVENUE, HACKENSACK, NJ, 07601

(21) Appl. No.: 09/932,257

(22) Filed: Aug. 17, 2001

Related U.S. Application Data
(60) Provisional application No. 60/226,340, filed on Aug. 18, 2000.
(60) Provisional application No. 60/226,339, filed on Aug. 18, 2000.

(51) Int. Cl. A61K 39/00; C12N 9/00; C07H 21/04; C12P 21/02; C12N 5/06

(52) U.S. Cl. 424/185.1; 435/69.1; 435/183; 435/320.1; 435/325; 536/23.2

(57) ABSTRACT

A novel protein, MCEF, antibodies thereto, nucleic acid sequences that code for it, and probes for leukemia-associated translocation junctions; also process of using MCEF or other P-TEFb proteins such as CDC37, and HSP-90 to identifv reagents promoting dissociation of those proteins from each other or inhibiting their association and to discover anti-HIV reagents.

### CROSS-REFERENCE TO RELATED APPLICATIONS

This application claims priority of U.S. Ser. No. 60/226,340, filed Aug. 18, 2000 and U.S. Ser. No. 60/226,339, filed Aug. 18, 2000, the contents of both of which are incorporated by reference in their entireties.

### RESEARCH SUPPORT

The research leading to the present invention was supported, at least in part, by AIDS grant #AI37327-05 from the National Institutes of Health. Accordingly, the Government may have certain rights in the invention.

### BACKGROUND OF THE INVENTION

Eukaryotes use three polymerases (Pol), named Pol I, II and III [1]. Eukaryote transcription can be regulated at multiple levels, including access to chromatin, formation of a pre-initiation complex, initiation, promoter clearance, elongation and termination (Reviewed in: [2]). For Polymerase II (Pol II) transcribed sequences, such as those of HIV-1, it is well established that hyperphosphorylation of the Pol II-Carboxy Terminal Domain (CTD), by the general transcription elongation factor termed Positive Transcription Elongation Factor b (P-TEFb), is a regulatory event affecting elongation (Reviewed in: [3]).

For HIV-1, the viral protein Tat regulates transcription elongation by recruiting/stabilizing P-TEFb. Tat accomplishes this by interacting in an apparent ternary complex between P-TEFb and a transcriptionaly nascent RNA stem-loop structure termed TAR, resulting in the phosphorylation of the Pol II CTD, by the CDK9 component of P-TEFb ([4-6]; Reviewed in: [3]. Tat transactivation is key to the high levels of HIV-1 replication in vivo, resulting in AIDS. The ability to block Tat transactivation by targeting Tat or any of the cellular components involved in Tat transactivation, such as P-TEFb, is an area of intense research [3]. However, the exact composition of P-TEFb remains unknown [7].

The term P-TEF was coined by Marshall and Price in 1992, for a theoretical factor in drosophila nuclear extracts, that was required for efficient transcription elongation, and was sensitive to the nucleotide analog DRB [8]. Subsequent chromatographic fractionation of drosophila P-TEF resulted in purification of a transcription elongation activity termed P-TEFb, comprising stoichiometric 43 kD and 124 kD bands on SDS-PAGE [9]. P-TEFb was then found to contain a novel Pol II CTD kinase activity [10]. Similarly, a Tat Associated Kinase (TAK) activity from human cells, was found to hyperphosphorylate the Pol II CTD [11]. TAK was later found to be the human equivalent of P-TEFb, and SDS-PAGE bands of 40 kD, 87 kD, 105 kD, 133 kD, 140 kD and 207 kD were shown for some human P-TEFb/TAK preparations ([12]; Reviewed in: [7] and in [3]), whereas bands of 43 kD, 49 kD, 55 kD, 61 kD, 68 kD, 87 kD, were shown for other P-TEFb preparations [13]. The difference in band sizes for the P-TEFb components isolated by different groups, may be the result of methodology differences. In particular, the choice of cell lines from which P-TEFb is purified, chromatographic versus immunopurification of P-TEFb, and the salt concentrations during the purification steps, may be responsible for the observed differences.

The 43 kD band for drosophila P-TEFb, present in all preparations, was identified as CDK9 (formerly known as PITALRE) [12], as was the kinase subunit from human P-TEFb [14]. The upper 124 kD drosophila P-TEFb band was identified as Cyclin T1 [15], as was the upper 87 kD band in human P-TEFb [4]. Cyclin T2A and T2B were later found to also interact with CDK9 in P-TEFb [16].

Activation by P-TEFb requires a DRB Sensitivity Inducing Factor (DSIF), whose target of action is the human homologue of yeast SPT5 (hSPT5), referred to as DSIFp160, and associated proteins [17, 18].

We sought to identify all the components of P-TEFb and DSIF. In order to address this question thoroughly, we have made stable cell lines expressing FLAG-tagged components of P-TEFb and DSIF. From stable FT-CDK9-expressing and FT-DSIFp160-expressing HeLa-3S cell lines, we immunopurified large quantities of P-TEFb and DSIF. The identity of the specific protein components of P-TEFb and DSIF were determined in an unbiased manner by direct sequencing. The DSIF complex was found to contain only DSIF-p14 (hSPT4) and DSIF-p160 (hSPT5). The P-TEFb complex was found to comprise Cyclin T1, T2A, T2B, CDK9, in accordance with previous publications (see above). In addition, pertinent to the present invention, the P-TEFb complex was found to contain the well known kinase chaperone pair HSP90-CDC37 [19] and a novel protein. A molecular clone of 5,858 bp, corresponding to this novel protein, was obtained for a novel gene, located on chromosome 5, near the interleukin gene cluster, within Alu repeats. We name this gene the MCEF gene, for Major CDK9-complex Elongation Factor. The MCEF gene codes for a serine/proline rich protein that is expressed as a major 10 kb transcript in human heart, brain, placenta, liver, skeletal muscle, kidney, pancreas, spleen, lymph nodes, thymus, peripheral blood leukocyte, bone marrow, and fetal liver.

Intriguingly, we find phylogenetically that MCEF is a novel member of the AF4/FMR2 family of transcription factors [20]. The most frequent chromosome rearrangement in childhood acute lymphoblastic leukemias (ALL) involves a chromosome 4-11 translocation resulting in a chimeric protein between the MLL gene product and the AF4 gene product [21, 22]. Chromosome 5-region fragility has been reported in ALL [22]. We propose that MCEF is a candidate gene for these translocations. Furthermore, we propose that therapies aimed against P-TEFb may have therapeutic benefit against these leukemias.

This work identifies a novel gene, mRNA, cDNA and proteins, likely involved in leukemia, namely, the MCEF gene, MCEF mRNA, MCEF cDNA and MCEF proteins and proposes that MLL-MCEF translocations can recruit P-TEFb as the mechanism causing at least certain forms of leukemia. Thus therapies against P-TEFb are candidates for benefitting patients with this form of leukemia.

Also, this work identifies a novel target for potential anti-P-TEFb, anti-Tat-P-TEFb, or antiHIV therapies, namely: HSP90, CDC37 and MCEF, and identifies all of the components of P-TEFb, and recognizes that therapies against certain forms of leukemias, involving the AF4/FMR2 family members may be beneficial against HIV infection and AIDS.

O'Keefe et. al. have published that the components of human P-TEFb consist of the previously known CyclinT1-CDK9 pair, as well as HSP90, HSP70 and CDC37 [23]. Taki et. al. have published a base sequence of a cDNA for a theoretical MLL-fusion protein derived from an ALL patient [24], which corresponds to a novel theoretical chromosome 5 gene they term AF5 that, as regards the open reading frame (ORF) of its DNA. The relationship of that published sequence to the one shown in the present application is discussed below.

The mechanism of action for MLL-AF4 remains unknown. The finding that MCEF immunopurifies with FT-CDK9, suggests that MLL-MCEF fusions would have the potential to recruit P-TEFb to MLL DNA-binding domain target genes. We propose that a similar mechanism of P-TEFb recruitment similar to that by Tat for HIV, could be recapitulated by MLL-MCEF (or MLL used with other AF4 family members) in ALL with chromosome 5 instability (or instabilities involved in translocations between other AF4 family members and MLL). Thus therapies aimed against P-TEFb components could benefit ALL patients as well. These would include therapies against all the components of P-TEFb: HSP90, CDC37, MCEF,Cyclin T1, Cyclin T2A, Cyclin T2B, and CDK9.

### SUMMARY OF THE INVENTION

In a general aspect, the invention is a preparation comprising an MCEF-type polypeptide or an MCEF homolog, the preparation being substantially free of other human proteins.

For embodiments of this invention described below, an MCEF-type polypeptide (or an MCEF-type protein) is one that comprises at least 50 consecutive amino acids of the amino acid sequence of mammalian MCEF, said amino acid sequence being the one encoded by the MCEF cDNA insert contained in the plasmid deposited by ATCC accession number PTA-1502, and an MCEF homolog is a sequence of length equal to an MCEF-type polypeptide, and having at least N percent identity with that polypeptide, N being 70, preferably 80, more preferably, 90.

Percent identity is calculated by aligning the peptide sequence and its homolog end-to-end and measuring the percent of amino acids that are identical. To illustrate, consider the following hypothetical peptide (first line) and its homolog (second line), where the letters A, G, and T, stand for amino acids (and the amino acid sequences are referred to as SEQ ID NO:28 and SEQ ID NO:29, respectively).

AAAAAAAAAATTTTTTTTTT

AAAAAAGGGGGGTTTTTTT

At the twenty positions along the sequences, there is identity at 14. Therefore N is 70. The same approach is used to calculate the percent identity between a base sequence and its homolog, where the letters, A, G, and T stand for bases (and the base sequences are referred to as SEQ ID NO:30 and SEQ ID NO.:31, respectively).

One preferred MCEF-type protein for all aspects of this invention is one with the complete amino acid sequence encoded by the cDNA insert contained in the plasmid deposited under ATCC accession number PTA-1502.

The MCEF-coding region of the plasmid deposited under ATCC deposit number PTA-1502 is believed to be that shown in FIG. 4A, at greater than 99% accuracy. Its relation to the sequencing and that of Taki et al., Proc. Natl. Acad. Sci. USA, vol. 96, 14535-14540 (1999) is discussed below in the summary of that Figure.

The terms “MCEF-type polypeptide” and “MCEF-type protein” are used interchangeably, smaller molecules more likely to be described by as a polypeptide, those of 55 kD or more tending to be described as a protein.

In another aspect, the invention is an MCEF-type polypeptide or MCEF homolog which has been synthesized by recombinant DNA techniques, a recombinant DNA technique being one in which MCEF nucleic acid coding for said polypeptide is added to a host cell that is not a human cell, (preferably the host cell is a microorganism such as E. Coli, but more preferably an insect cell such as a Spodoptera Frugiterda SF21 cell) so as to result in the synthesis of the MCEF-type polypeptide or the homolog. A preparation of an MCEF-type polypeptide made by recombinant techniques in a non-human cell in which the only human cDNA was for MECF is an example of a preparation that is substantially free of other human proteins. The MCEF-type polypeptide or the homolog can be purified away from host cell proteins by immunoprecipitation with anti-MCEF antibodies but also by other standard protein purification techniques such as electrophoresis in one or two dimensions and chromatography.

In another aspect, the invention is a polypeptide comprising an immunogenic fragment of the MCEF-type protein encoded by the cDNA insert contained in the plasmid deposited under ATCC accession number PTA-1502. Such a fragment can be as small as 7 amino acids, but is preferably at least 50 amino acids in length. A fragment is immunogenic if it induces antibodies (in a mammal, especially 60 days after i.p. injection of 4 successive doses of the molar equivalent of 100 μg complete MCEF protein into rabbits at intervals of 1.5 weeks between doses) that are reactive with the complete MCEF-type protein encoded by the cDNA insert contained in the plasmid deposited under ATCC accession number PTA-1502. However, it is preferred to have a sequence of at least 100 amino acids for such purposes, and most preferably at least 250 amino acids.

The invention is also a process of purifying an MCEF-type protein, said process comprising the steps of:

(a) isolating a P-TEFb complex (preferably by immunoprecipitation); and

(b) purifying the MCEF-type protein from the P-TEFb complex (by any biochemical technique including but not limited to electrophoresis, 2-dimensional electrophoresis, chromatography and immunoprecipitation). In another aspect, the invention is a preparation of an MCEF-type protein purified by such a process.

In another general aspect, the invention is an isolated MCEF nucleic acid molecule comprising (a) an MCEF base sequence that is at least 150 consecutive bases of, but preferably all of, the MCEF-coding base sequence of the cDNA insert in the plasmid deposited under ATCC accession number PTA-1502, or (b) a base sequence that is code-wise degenerate with such a MCEF base sequence, or (c) a base sequence that is a homolog of at least N per cent identity of such an MCEF base sequence, N being 70 but preferably 80 and more preferably 90. A nucleic acid molecule that is in a preparation of molecules identical to itself is considered an isolated molecule. A nucleic acid molecule free of other nucleic acid molecules is an isolated nucleic acid molecule. A nucleic acid molecule that is free of other human nucleic acid molecules is an isolated molecule. An isolated nucleic acid molecule would also include a recombinant DNA molecule such as a plasmid or vector containing sequences that are necessary for the molecule to be replicated or expressed in a non-human organism. As a result, the plasmid deposited under ATCC deposit number PTA-1502 is an example of an isolated MCEF nucleic acid molecule.

The phrase “isolated MCEF nucleic acid molecule” does not include DNA molecules that are the entire DNA complement of the any mammalian (especially, human) chromosome. It also does not include a DNA molecule that comprises all the naturally occurring MCEF exons and introns that together make up the gene whose expression results in mammalian cells synthesizing the complete amino acid sequence encoded by the cDNA insert contained in the plasmid deposited under ATCC accession number PTA-1502.

The base sequence of the ATCC nucleic acid deposit having accession number PTA-1502 has been determined by us to be that shown in FIG. 4A. However, the base sequence in FIG. 4A is not identical to a sequence published by Taki et al.Proc. Natl. Acad. Sci. USA, vol. 96, 14535-14540 (1999) for a protein that is, according to our nomenclature, an MCEF-type protein. It is currently believed by us that said base sequence in Taki et al. is at least in part more correct (up to a maximum of 17 bases in 5858,or 0.3%) than that in FIG. 4A. In all, 17 differences with the Taki nucleic acid sequence and MCEF sequence occur. In addition, the MCEF sequence lacks 292 bases present in the Taki et al. publication sequence at the 5′-end and the Taki sequence lacks 1,894 bases present in the MCEF sequence at the 3′-end.

In a related aspect the invention is a hybridization detection process for detecting MCEF nucleic acid, said process comprising the steps of:

a) hybridizing an MCEF hybridization probe against target nucleic acid (RNA or DNA) so as to form a hybrid of said probe and said target; and

b) detecting said hybrid. The MCEF hybridization probe comprises an MCEF nucleotide sequence or a nucleotide sequence complementary to an MCEF nucleotide sequence, or a homolog of at least N percent identity to either of said sequences, said MCEF nucleotide sequence comprising at least M contiguous nucleotides of the nucleotide sequence of the cDNA insert in the plasmid deposited as ATCC accession number PTA-1502, wherein M is 10 (preferably 15, more preferably 20) and wherein N is 70 (but preferably 80, more preferably 90).

In a related aspect the invention is a hybridization translocation detection process for a leukemia chromosomal translocation, said process comprising the steps of:

a) hybridizing an MCEF hybridization probe either against target nucleic acid spanning a translocation junction (preferably mRNA spanning a translocation junction) from a person or against nucleic acid generated by amplification procedures (e.g. PCR, in which case the probe functions as a primer for nucleic acid synthesis) dependent on such a target nucleic acid as a template; and

b) detecting said hybrid. The detection of the hybrid will contribute to a diagnosis that the person has leukemia associated with a chromosomal translocation. The MCEF hybridization probe comprises an MCEF nucleotide sequence or a nucleotide sequence complementary to an MCEF nucleotide sequence, or a homolog of at least N percent identity to either of said sequences, said MCEF nucleotide sequence comprising at least M contiguous nucleotides of the nucleotide sequence of the cDNA insert in the plasmid deposited as ATCC accession number PTA-1502, wherein M is 10 (preferably 15, more preferably 20) and wherein N is 70 (but preferably 80, more preferably 90).

For any hybridization assay described herein, the hybridization probe may be detectable because it has a detectable label, such as a flourescent dye, a radioactive isotope or a component of a detectable biotin-strepavidin pair, or alternatively a nucleotide sequence that can be accessed by another nucleic probe that is detectable or part of a detectable system. More preferably the probe is detected by virtue of the fact that it can trigger an amplification process, such as a primer for PCR (polymerase chain reaction) the products of which amplification are detectable.

Detection of a translocation mRNA molecule may be accomplished by using a probe that will only hybridize to a base sequence spanning the translocation junction in the mRNA molecule but more preferably by using PCR amplification techniques wherein one primer is specific for a sequence on one side of the junction and the other primer is specific for a sequence on the other side of the junction. The target nucleic acid may, for example, be part of a purified nucleic acid preparation, be chromosomal within a cell or a nucleus, or be a purified chromosomal preparation. The fact that a chromosome that hybridizes to the MCEF hybridization probe is one that has undergone translocation can be demonstrated by showing it also hybridizes to a second probe, one that is specific for a chromosome that normally does not hybridize to MCEF and that has a different detectable label (e.g., a different color) than the MCEF probe.

For any hybridization assay described herein, any nucleic acid probe that can be substituted for an MCEF nucleic acid molecule as a probe and still detect the same target nucleic acid molecules as an MCEF nucleic acid molecule is an aspect of the invention as are the above-noted hybridization processes if such a substitute probe is used. Such substitute probes include a DNA probe molecule, or an RNA equivalent thereof, said DNA probe molecule between 14 and 70 nucleotides in length, which DNA probe molecule forms a hybrid with a DNA target molecule consisting of the base sequence of the cDNA insert in the plasmid deposited under ATCC accession number PTA-1502, more preferably consisting of the coding sequence of said cDNA, or with a complement thereof, at a temperature 10° C. below the Tm calculated according to the following equation

Tm=81.5−16.6(log₁₀[Na+])+0.41(%G+C)−(600/N)

where N is the chain length in nucleotides and where the hybridization is done in aqueous solution with the Na+ concentration at 1 M, and wherein the G+C content refers to the probe.

In another general aspect, the invention is a reagent binding assay for identifying a reagent that binds to an MCEF-type polypeptide, said process comprising the steps of:

a) reacting the reagent with the MCEF-type polypeptide; and

b) observing an affinity of the reagent for the MCEF-type polypeptide; wherein the MCEF-type polypeptide is preferably one with the complete amino acid sequence encoded by the cDNA insert contained in the plasmid deposited under ATCC accession number PTA-1502.

In a related aspect, the process is a leukemia-directed reagent-binding assay, the process comprising the steps of (a) performing a reagent binding assay for a reagent that binds to a P-TEFb protein and (b) assaying for the effect of the reagent on the growth rate of leukemia cells (rate of cell division) wherein said P-TEFb protein is selected from the group consisting of an MCEF-type polypeptide, CDK9, CDC37, HSP90, Cyclin T1, Cyclin T2A and Cyclin T2B.

In another related aspect, the process is a leukemia-directed therapeutic process, the process comprising the steps (a) and (b) of the leukemia-directed reagent binding assay and then further comprising a step (c) of administering the reagent to a person suffering from leukemia.

In another aspect, the invention is a dissociation assay that is an assay for reagents that will disrupt a P-TEFb complex to generate a P-TEFb protein unassociated with other P-TEFb complex members, said assay comprising treating a P-TEFb complex with said reagent and determining whether the P-TEFb protein has been disassociated, wherein said P-TEFb protein is selected from the group consisting of an MCEF-type polypeptide, HSP90 and CDC37. Preferably the assay is done by subjecting the P-TEFb complex, both untreated and treated with the reagent, to immunoprecipitation dependent on antibodies against a P-TEFb protein and then determining which proteins are immunoprecipitated and in what amounts. The latter analysis can be done by Western Blot or silver staining analysis.

In another aspect, the invention is an association inhibition assay for reagents that interfere with the ability of a P-TEFb protein to associate with one or more proteins, other than itself, of the P-TEFb complex, said P-TEFb protein selected from the group consisting of an MCEF-like polypeptide, HSP90, and CDC37. Preferably, this is done by comparing immunopurified P-TEFb from untreated cells to immunopurified P-TEFb from cells treated with the reagent.

In a related aspect, the invention is the process of administering a drug to a patient suffering from leukemia, said drug selected from the group consisting of (a) drugs that bind a P-TEFb protein, (b) drugs that dissociate P-TEFb to generate an unassociated P-TEFb protein; and (c) drugs that interfere with the ability of a P-TEFb protein to associate with one or more P-TEFb proteins, other than itself, wherein a P-TEFb protein is selected from the group consisting of an MCEF-type polypeptide, CDK9, Cyclin T1, Cyclin T2A, Cyclin T2B, HSP90, and CDC37.

In another aspect, the invention is an MCEF-reactive antibody or serum, said antibody or serum being immunoreactive with an MCEF-like polypeptide.

In a related aspect, the invention is a diagnostic/prognostic assay wherein a sample of fluid, cells or tissue, said sample preferably blood or plasma, from a patient with leukemia is reacted with an antibody or serum reactive with a P-TEFb protein to form an antibody-protein complex, said protein being a P-TEFb protein or a fusion protein comprising at least 10 consecutive amino acids of an MCEF-type polypeptide, such that the greater the amount of said complex (especially if said protein is said fusion protein), the poorer the prognosis for the patient in regard to leukemia, wherein the P-TEFb protein is selected from the group consisting of an MCEF-type polypeptide, CDK9, Cyclin T1, Cyclin T2A, Cyclin T2B, HSP90 and CDC37.

In another general aspect of the invention, the process is a reagent-binding assay for identifying a reagent that binds to a P-TEFb protein and that is capable of interfering with HIV transcription or HIV replication, said process comprising the steps of:

a) reacting the reagent with the P-TEFb protein;

b) observing an affinity of the reagent for the P-TEFb protein;

c) adding the reagent to an assay for HIV transcription or HIV replication or said complex;

d) observing a decrease in the amount or transcript length in the assay for HIV transcription or observing a decrease in the amount of HIV replication in the assay for HIV replication;

wherein the P-TEFb protein is selected from the group consisting of an MCEF-type polypeptide, HSP90, and CDC 37.

In a related aspect, the process is a therapeutic process, the process comprising the steps (a) through (d) of the reagent binding assay and then further comprising a step (5) of administering the reagent to a human subject suffering from HIV infection (or AIDS).

In another aspect, the invention is a therapeutic process comprising the steps of:

(1) identifying a reagent as one that dissociates P-TEFb so as to generate an unassociated P-TEFb protein, said P-TEFb protein being selected from the group consisting of an MCEF-like polypeptide, HSP90, and CDC 37.

(2) administering the reagent to a person who has AIDS or is HIV-infected.

Preferably step (a) of the assay is done by subjecting the P-TEFb complex, both untreated and treated with the reagent, to immunoprecipitation dependent on antibodies against a P-TEFb protein and then determining which proteins are immunoprecipitated and in what amounts. The latter analysis can be done by Western Blot or silver staining analysis.

In another aspect, the invention is a therapeutic process comprising the steps of:

(a) identifying a reagent as one that interferes with the association of a first protein, with a second protein, said first protein selected from the group consisting of an MCEF-like polypeptide, HSP90, and CDC 37, said second protein selected from the group consisting of CDK9, cyclin T1, cyclin T2A, cyclin T2B, HSP90, and CDC37, and an MCEF-type polypeptide, provided said second protein is not the same as the first protein (e.g., the first and second proteins cannot both be a CDC37 protein); and

(b) administering the reagent to a person who has AIDS or is HIV-infected. Preferably step (a) is done by comparing immunopurified P-TEFb from untreated cells to immunopurified P-TEFb from cells treated with the reagent.

In a related aspect, the invention is the process of administering a drug to a patient who is HIV-infected or suffers from AIDS, said drug selected from the group consisting of (a) drugs that bind to a P-TEFb protein, (b) drugs that dissociate P-TEFb to generate unassociated P-TEFb protein, and (c) drugs that interfere with the ability of the P-TEFb protein to associate with one or more proteins, other than itself, of the group consisting of CDK9, Cyclin T1, Cyclin T2A, Cyclin T2B, HSP90, and CDC37, and an MCEF-type polypeptide, and wherein the P-TEFb protein is selected from the group consisting of an MCEF-type polypeptide, HSP90, and CDC37.

In a related aspect, the invention is an assay system for testing the effects of a reagent on a P-TEFb protein's role in HIV transcription, said system comprising 3 components:

(a) DNA comprising the TAR region of HIV DNA (from the viral HIV LTR) and further comprising a transcribable region (i.e., is transcribed in the presence of a polymerase);

(b) A DNA-dependent RNA poymerase; and

(c) purified P-TEFb protein or recombinant P-TEFb protein, such that components (a), and (b) and (c) are optionally in HIV-infected cells, wherein the P-TEFb protein is selected from the group consisting of an MCEF-type polypeptide, HSP90, and CDC37.

In a related aspect the invention is an MCEF nucleic acid hybridization assay for nucleic acid samples from individuals that are HIV-infected or AIDS-afflicated, said process comprising the steps of:

a) hybridizing an MCEF hybridization probe preparation against target nucleic acid from a person or against nucleic acid generated by amplification procedures (e.g. PCR) from a person, said person being HIV-infected or AIDS-afflicted; and

b) quantifying the amount of hybridized MCEF.

wherein the MCEF hybridization probe comprises an MCEF nucleotide sequence or a nucleotide sequence complementary to an MCEF nucleotide sequence, or a homolog of at least N percent identity to either of said sequences, said MCEF nucleotide sequence comprising at least M contiguous nucleotides of the nucleotide sequence of the cDNA insert in the plasmid deposited under ATCC accession number PTA-1502, where M is 10 (preferably 15, more preferably 20) and wherein N is 70 (but preferably 80, more preferably 90). Preferably the amount found in (b) is compared to the amount found in (b) with a person not HIV-infected or AIDS afflicted.

The hybridization probe may be detectable because it has a detectable label, such as a flourescent dye, a radioactive isotope or a component of a detectable biotin-strepavidin pair, or alternatively a nucleotide sequence that can be accessed by another nucleic probe that is detectable or part of a detectable system. More preferably the probe is detected by virtue of the fact that can trigger an amplification process, such as a primer for PCR (polymerase chain reaction) the products of which amplification are detectable.

In another aspect, the invention is an immunologic assay that comprises as step wherein a sample of fluid, cells or tissue, said sample preferably blood or plasma, from a patient who is HIV-infected or AIDS afflicted, is reacted with an antibody or serun reactive with a P-TEFb protein to form an antibody-protein complex, said protein being a P-TEFb protein.

The reagent binding assay is useful for identifying reagents that will affect HIV levels in infected patients. The therapeutic processes are useful for treatment of HIV infection or AIDs. The assay for a reagent's effect on a P-TEFb protein's role in HIV transcription is useful for the anti-HIV drug making process and for studying the mechanism of HIV transcription.

The hybridization assay is useful for monitoring the levels of mRNA of proteins involved in a pathological process, HIV infection or AIDs. The immunologic assay is useful for monitoring specific protein levels in those diseases.

Antibodies against MCEF-type polypeptides are useful as diagnostic tools in regard to leukemia or AIDS and also as a means of purifying such peptides. They are also useful as an analytical reagent for detecting such peptides in experiments studying the mechanism of transcription. Sera against such peptides are useful as a source of such antibodies.

The MCEF-type polypeptides (and therefore processes of making them) and homologs are useful for the production of antibodies against such peptides and are also usefill to isolate reagents that interfere with transcription. Assays that seek binding agents that interfere with transcription are useful for both the study of transcription generally and for screening for therapeutic agents where effects on transcription are desired.

Nucleic acids coding for MCEF-type polypeptides (and hybridization processes for nucleic acids coding for such peptides) are useful for directing the synthesis of MCEF-type polypeptides for detecting clones that produce cDNA for MCEF-type polypeptides and for detecting chromosome translocations that comprise base sequences coding for MCEF-type polypeptides; and for determining related mRNA levels in cells. The hybridization assay is also useful for monitoring the effects of levels of related mRNA of proteins involved in a pathological process. The immunological assays are also of value in monitoring specific protein levels in such pathological processes. HIV infection or AIDs and leukemia are examples of pathological processes, but other relevant pathological processes are also contemplated.

Assays that seek binding agents that interfere with transcription are useful for both the study of transcription generally and for screening for therapeutic agents where effects on transcription are desired. Nucleic acids coding for MCEF-type polypeptides are useful for directing the synthesis of MCEF-type polypeptides for detecting clones that produce cDNA for MCEF-type polypeptides and for detecting chromosome translocations that comprise base sequences coding for MCEF-type polypeptides and for determining related mRNA levels.

Assays for reagents that bind to P-TEFb proteins are useful for identifying reagents that allow studies on the effect of P-TEFb protein on transcription. In particular, proteins in assays for isolating reagents that have an effect on leukemic cell growth are of value in the discovery process of anti-leukemic drugs.

### BRIEF DESCRIPTION OF THE DRAWINGS

FIG. 1. Establishment of stable MCE-HeLa-3S-FT-(X)-expressing cell lines. A) Schematic representation of experimental strategy. A FT-linker was used to clone the respective PCR products (X) into the mammalian IRES expression vector pCIN4, thus generating the pMCE-CIN4-FT-(X)-constructs. These constructs were then used to generate the stable MCE-HeLa-S-FT-(X)-stable cell lines as confirmed by WB (see FIG. 1B). From these cell lines nuclear and S-100 extracts were prepared and complexes immunopurified via the FLAG tagg. B) WB with FT-antibody, used to select clones SPT5-8, CDK9-7 and CDK9M-9. Lanes 1-8, respective whole cell extracts from clones. Lane 9 HeLa-S3 control. Lane 10 BR MW standards (BRL).

FIG. 2. Immunopurified FT-CDK9 from the MCE-HeLa-S3-FT-CDK9-7 stable cell line contains CTD and DSIFp160 kinase activity. A) WB with FT-antibody throughout immunopurification steps. Lanes 1, 2, 4, 7, controls. Lane 3, nuclear extract from the MCE-HeLa-S3-CDK9-7 cell line. Lanes 5, 6, complexes immunopurified in BC buffer containing 270 mM KCl, 0.1% NP-40, from the MCE-HeLa-S3-CDK9-7 and the MCE-HeLa-S3-DSIFp160-8 cell lines respectively. Lanes 8, 9, same as 5, 6, but immunopurified in BC buffer containing 500 mM KCl. B) WB with CDK9 antibody. Lane 1, immunopurified FT-CDK9-7 complex. Lane 2, HeLa-3S nuclear extract. Lane 3, MCE-HeLa-S3-CDK9-7 cell line nuclear extracts. C) Immunopurified FT-CDK9 can phosphorylate the CTD of Pol II and DSIFp160. Lane 1, 2, 3, 4, 5, control reactions. Lanes 5-12, the indicated combinations of substrates are incubated with either FT-CDK9 (5-8), or FT-CDK9M (9-12).

FIG. 3. Immunopurified P-TEFb is composed of HSP-90, CDC-37, Cyclin T, CDK9 and a novel component. A) Silver stained gel of the complex immunopurified from nuclear extracts of MCE-HeLa-S3-CDK9-7 at 270 mM (Lane 2), 350 mM (Lane 4) and 500 mM (lane 6), or from S-100 extracts at 500 mM (Lane 8). Lanes 1, 3, 5, 7, control vector cell line extracts. Lane 9, molecular weight standards. Components identified by direct sequencing are indicated. Non-specific components are labeled 1-7 and were not sequenced. B) As in A, but for MCE-HeLa-S3-DSIF-p160-8. C) Comparison of the complexes immunopurified at 100 mM or 500 mM KCl, for FT-CDK9M (Lanes 1, 2) versus FT-CDK9 (Lanes 3, 4) and controls (Lanes 5, 6). Lane 7, molecular weight standards.

FIG. 4. Molecular cloning and characterization of the novel P-TEFb component MCEF. A) A 5,858 bp MCEF cDNA sequence is indicated and numbered on the left hand margin (SEQ ID NO:2). A putative protein translation of a major theoretical ORF is indicated using single letter amino acid codes, numbered on the right hand margin in black (SEQ ID NO:3). The TGA start codon is at position 66-68. Underlined indicated GSP-3 (372-393) and GSP-4 (402-457) primers from 5′ to 3′ respectively. The lower case letters within GSP-4, indicate a Bsu36I site (436-443). The complement of GSP-1 (595-569) and GSP-2 (557-529) primers, going from 3′ to 5′, are underlined. A BglII site (270-275) and a NcoI site (841-846) are underline and delimit the 570 base pair fragment which was used for screening the Zap library and as a probe for northern blot in FIG. 4B. B) Northern blot using the probe indicated in FIG. 4A (272-843). The tissues examined are indicated above the lanes and MW standards to the left of figure. A major 10 kb transcript is indicated by the arrow and minor transcripts are indicated by lines under arrow.

The base sequences and the amino acid sequences represent that published by Taki et al except for the following changes:

| base | base in | base in | amino acid | amino acid |
|---|---|---|---|---|
| position | this Figure | Taki et al. | this figure | Taki et al. |
| 855 | G | A | G | E |
| 1141 | T | C | I | T |
| 1213 | G | A | G | N |
| 1308 | G | A | S | S |
| 1399 | G | A | G | E |
| 1915 | T | C | F | S |
| 3239 | T | C | S | P |
| 3304 | T | G | G | V |

Additionally, bases 1-292 in Taki etal., which occur before the gene, are omitted here. Also, bases 3964-5858, shown here, are not disclosed in Taki et al.

FIG. 5. Effect of MCEF on HIV-1 LTR-directed transcription. A) ectopic expression via transient co-transfection of pcDNA-MCEF and pHIV-LTR-luc, versus co-transfection of pcDNA vector without MCEF. The figure shows an effect of the pcDNA-MCEF on the level of luciferase units measured. B) Control curve for an assay that is used to measure the effects of ectopic expression of MCEF on Tat trans-activation.

FIG. 6. HeLa-s cells express a p140 and p55 kD MCEF proteins. Western blots using anti-sera from rabbits injected with 3 different portions of recombinant MCEF protein. Two rabbits were used for each portion. Two were injected with either rMCEF1-715 (lanes 2-5), two with rMCEF1-530 (lanes 6-9), and two with rMCEF 1-420 (lanes 10-13). Pre-immune sera (P) and immune sera (I) were tested in western blots against HeLa-S nuclear extracts. A prominent band at a position of approximately 140 kD (p140) is seen in the immune lanes 3, 5, 7, 9, 11. A prominent band of 55 kD (p55) is seen in lane 13, and also in lanes 5 and 11. A fainter band at 55 kD is also seen in the other immune lanes. Note: lanes (strips) are not horizontally perfectly aligned, so the p 55 kD band appears lower or higher depending on the lane. Because the separation is greater between bands at the bottom of the gel, this effect is more pronounced for the p55 band than the p140 band.

FIG. 7: Model. Schematic representation of P-TEFb recruitment by Tat-Tar, versus proposed P-TEFb recruitment via MLL-MCEF fusions.

FIG. 8. Proof that MCEF is a component of P-TEFb. Western blot with anti-CDK9, showing immunoprecipitation of P-TEFb components with rabbit #823 and #828 antisera directed against rMCEF 1-715 and 1-300 respectively (lanes 1 and 3) versus no P-TEFb immunoprecipitation with pre-immune antisera of the same rabbits (lanes 2 and 4).

### DETAILED DESCRIPTION OF THE INVENTION

Abbreviations and glossary

FT stands for FLAG-tagged; i.e. tagged with the FLAG epitope.

FLAG stands for the 8-amino acid peptide, Asp-Tyr-Lys-Asp-Asp-Asp-Lys.

Pol II stands for DNA-dependent RNA polymerase II.

DRB stands for 5,6-dichloro-1-_-D-ribofuranosylbenzimidazole.

HIV-1 stands for human immunodeficiency virus type 1.

AIDS stands for Acquired Immune Deficiency Syndrome.

LTR stands for long terminal repeat.

r as the first letter of a protein name stands for recombinant.

kD stands for kilodalton.

GST stands for glutathione-s-transferase.

CTD stands for carboxyterminal domain.

Transactivation is a process whereby the product of one gene increases the expression of another gene.

Code-wise degenerate means that a base of a codon is changed without changing the amino acid that the codon codes for.

A “coding sequence” or a “coding region” of a nucleic acid for a designated protein refers to a region in an mRNA molecule that contains the base sequence which is translated into an amino acid sequence (i.e., that encodes that amino acid sequence), it covers any DNA or RNA sequence that is complementary in base sequence to such an mRNA sequence, it covers any DNA sequence that is the same as such an mRNA sequence (except that T is used in place of U) and, in the case of a DNA sequence that alternates introns with exons so that the sequence can be processed to make an mRNA molecule, “coding sequence” or “coding region” covers the populations of coding exons that determine the amino acid coding region of the mRNA molecule (i.e., that encode that amino acid sequence).

An RNA base sequence (or molecule) is equivalent to a DNA base sequence (or molecule) if they are identical except that U in the RNA base sequence replaced T in the DNA base sequence.

A kinase assay is a test for the ability of a protein or preparation of proteins to transfer phosphates to other proteins or proteins within a preparation of proteins SDS-PAGE stands for Sodium Dodecyl Sulfate Polyacrylamide Gel Electrophoresis.

RACE stands for random amplification of cDNA ends.

WB stands for western blot.

“P-TEFb components”, “P-TEFb complex members” and “P-TEFb proteins” are MCEF, CDK9, CDC37, cyclin T1, cyclin T2A, cyclin T2B, and HSP90.

“HSP90” includes HSP90α and HSP90β. An invention directed at HSP90 may require or involve only one of HSP90α or HSP90β.

“Translocation junction” refers to junctions created by chromosomal translocations.

The following one letter codes are used to represent amino acids:

S-serine, T-threonine, N-asparagine, Q-glutamine, K-lysine, R-arginine, H-histidine, E-glutamic acid, D-aspartic acid, C-cystine, G-glycine, P-proline, A-alanine, I-isoleucine, L-leucine, M-methionine, F-phenylalanine, W-tryptophan, V-valine, Y-tyrosine, X-any amino acid.

The following one letter codes are used to represent nucleic acids:

A-adenine, C-cytosine, G-guanine, T-thymidine, R represents A or G, Y represents T or C, N represents any nucleic acid.

Materials and Methods

ATCC Deposit

A deposit of human cDNA MCEF of 5,858 b.p. cloned into the EcoR I site of plasmid pBluescript of 2,960 b.p., pBs-MCEF, has been made under the terms of the Budapest Treaty at the American Type Culture Collection, 10801 University Blvd., Manassaas, Va. 20110-2209 on Mar. 17, 2000 under Accession Number PTA-1502. All restrictions upon the availability of the deposited material will be irrevocably removed upon granting of a U.S. patent based upon this patent application. The Accession Number is referred to at the ATCC as the Patent Deposit Designation.

Electrophoresis in SDS-PAGE

Electrophoresis in SDS-PAGE electrophoresis was calibrated with protein markers, 200 Rabbit Skeletal Muscle Myosin 116.25 KD E. Coli Beta-Galactosidase; 97.4 KD Rabbit Muscle Phosphorylase B Protein; 66.2 KD Bovine Serum Albumin; 45 KD Hen Egg White Ovalbumin; 31 KD Bovine Carbonic Anhydrase; 21.5 KD Soybean Trypsin Inhibitor; 14.4 KD Hen Egg White Lysozyme; and 6.5 KD Bovine Pancrease Aprotinin. The composition of electrophoresis buffer was 12.5 mM Tris-HCl pH 8.3, 180 mM glycine, 0.1% SDS.

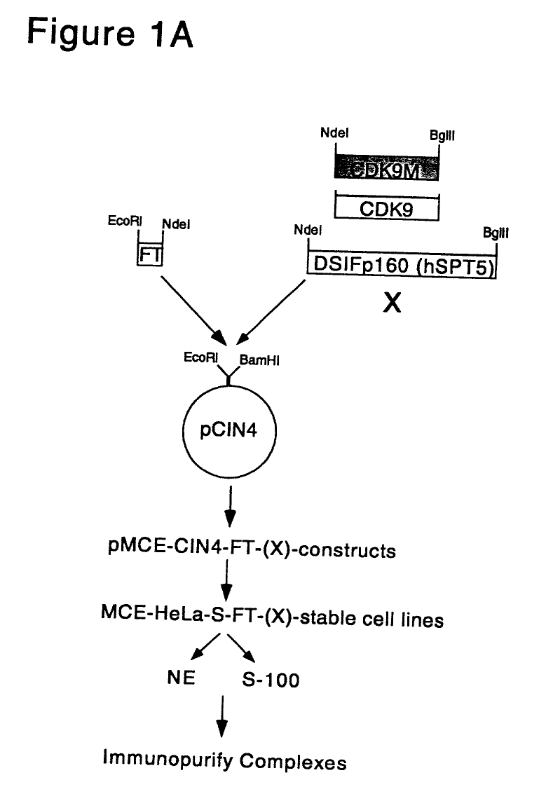

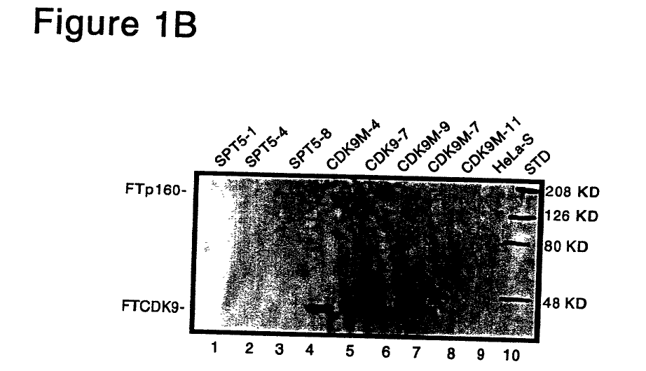

Making of stable HeLa-S3 cell lines expressing N-terminally-FLAG-tagged epitopes. The 1,118 bp CDK9/CDK9M [25] [4] and 3,317 bp DSIFp160 [26] cDNAs were amplified by PCR using primer pairs creating a 5′-NdeI site and a 3′-BglII site. Pairs were: MEK9NdeI Forward: 5′-CGGAATTCCATATGGCAA AGCAGTACGACTCG-3′(SEQ ID NO:4); MEK9BglII Reverse: 5′-GGAAGATCTTCCTCAGAAGACGGGCTCTTTGAG-3′(SEQ ID NO:5): MEp160NdeI Forward: 5′-CGGGAATTCCATATGTCGGACAG CGAGGACAGC-3′(SEQ ID NO:6); MEP160BglII Reverse: 5′-GGAAGATCTTCAGGCTTC-3′(SEQ ID NO:7). A FT linker was annealed from the primer pair: MEFTEcoRI Upper: 5′-AATTCATGGACTACAAAGACGATGACGATAAACAT-3′(SEQ ID NO:8); MEFTNdeI Lower: 5′-TATGTTTATCGTCATCGT CTTTGTAGTCCAT-3′(SEQ ID NO:9) generating a EcoRI-FT-NdeI linker. The FT linkers and respectively digested PCR products were ligated in 3-way reactions (10:1:1 molar ratio respectively) into the EcoRI and BamHI sites of pCIN4 (pIRESneo, Clontech) [27], generating the pMCE-CIN4-FT-(X)-constructs (FIG. 1). Junctions of constructs were sequenced on an Applied Biosystems automated fluorescent sequencer using primers MET7 Short: 5′-TAATACGACTCACTATAG-3′(SEQ ID NO:10) or MEInvT7:5′-CTATAGTG AGTCGTATTA-3′(SEQ ID NO:11). Adherent HeLa-S3 cells (ATCC# CCL-2.2) were grown and maintained in standard 37° C. CO2 incubators, in DMEM (JRH), supplemented with 10% BCS (JRH), 10 ml/L Pen. Strep. (Sigma # P-0906) in dishes. An 80% confluent 150 mm×25 mm dish of Hela-S3 cells was transfected using LipofectAMINE (GibcoBRL) as per the manufacturers suggested protocol, with either 5, 2.5, 0.5 μg of pCIN4 (for control cell line) or the same amounts of each pMCE-CIN4-FT-(X)-construct. At 48 hours post transfection, cells were subjected to selection at 500 μg/ml Geneticin (GibcoBRL). Clones expressing the neomycin resistance gene were selected over a period of 10 to 14 days, picked into individual plates and amplified under continued selection until confluent on 150×25 mm dishes. Clones were screened for expression of the FT-proteins by western blot with ANTI-FLAG M2 Monoclonal primary antibody (Sigma). Stocks for the newly created MCE-HeLa-S3-FT-(X)-stable cell lines were stored in liquid nitrogen.

Nuclear extracts. MCE-HeLa-S3-pCIN4-1, MCE-HeLa-S3-CDK9-7, MCE-HeLa-S3-CDK9M-9 and MCE-HeLa-S3-DSIFp160-8 clones were thawed and re-grown under selection (as above) until two confluent 150 mm×25 mm dishes, trypsinized and transferred to spinner flasks (re-adapting the HeLa-S3 derived cell lines to growth in suspension media) into Jokliks medium (JRH) containing 10% FBS, 10 ml/L Pen-Strep. Six liters of spinner flask grown cells were transferred to high density growth in a closed CO2 system and grown in DMEM (JRH) containing 10% FBS. Nuclear and S-100 extracts were prepared by the method of Dignam [28]. Extracts were either frozen directly in liquid nitrogen or pre-dialyzed to 100 mM KCl.

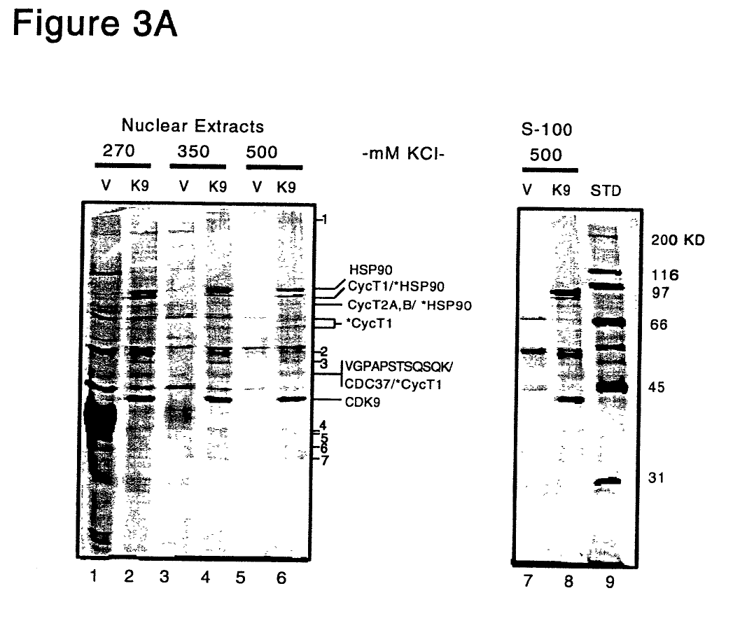

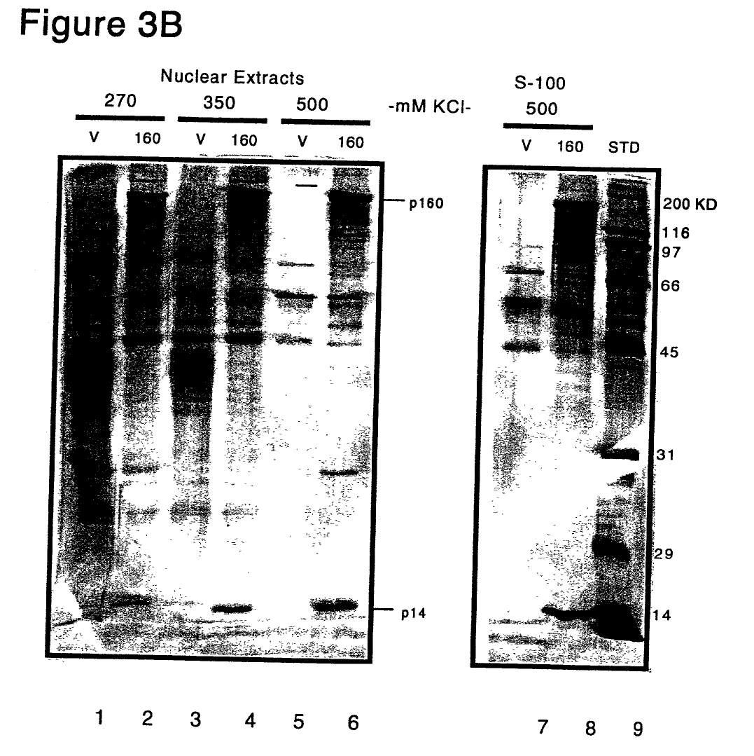

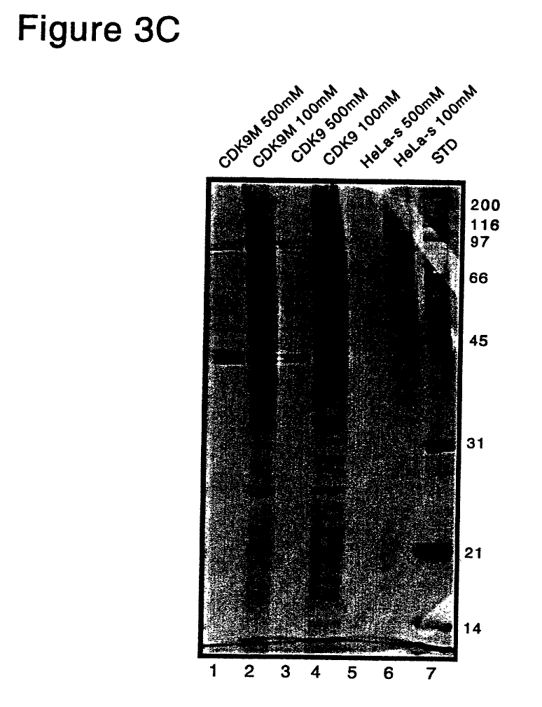

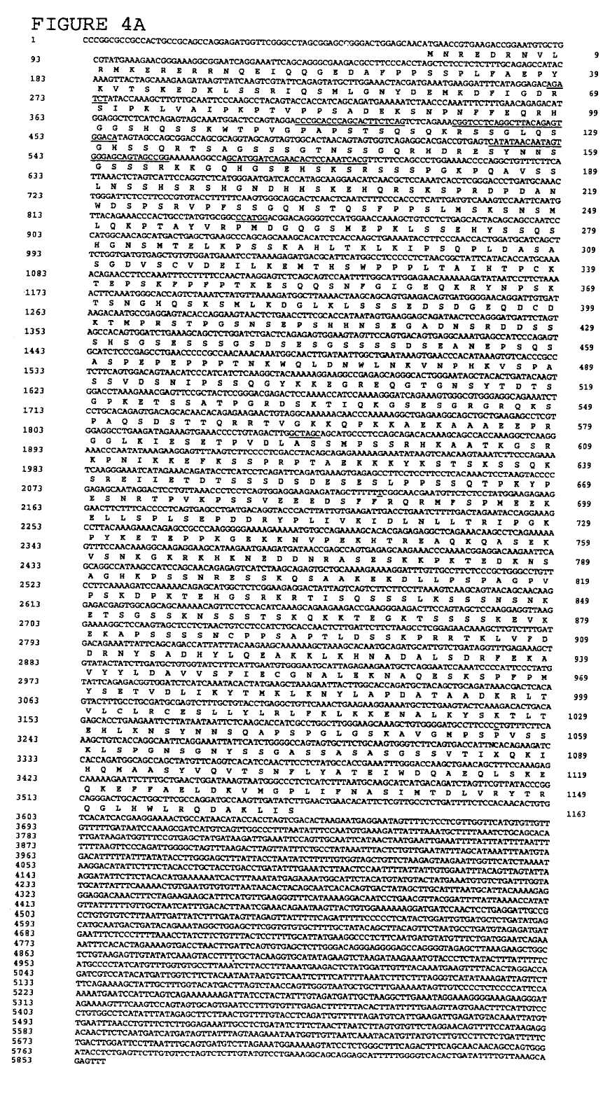

Immunopurification of P-TEFb and DSIF. Extracts were rotated at 4° C., 1 hour, in BC 100 buffer [28] containing 100 mM KCl with 0.1% NP-40 and M2-agarose. The respective M2-agarose-bound complexes were purified by centrifugation followed by 3 BC100 washes. Complexes were eluted with 200 ng/ml FT peptide, and snap-frozen stored in liquid nitrogen. Complexes were fractionated on SDS-PAGE and silver stained (KODAK), or coomassie-blue stained, or transfered to nitrocellulose membranes for western blotting, all by standard techniques. Complexes were confirmed to be P-TEFb and DSIF enzymatically by standard kinase assay and by peptide sequencing. Anti-CDK9/PITALRE antibody was purchased from Santa Cruz. M2-agarose anti-flag antibody (Kodak) was washed three times in BC buffer containing the respective concentration (see FIG. 3) of KCI, and 0.01% NP-40 and 1 mM DTT. Supernatant was cleared during the washes by centrifuging at 6K RPM for 2 minutes. For small scale binding, 1-5 ml of extracts in the same BC buffer KCL concentrations as the M2-agarose were incubated at 4° C., with rotation for one hour, with 20 ul of M2-agarose beads. To elute and purify the Flag-tagged complexes captured by the beads, the beads were first washed with the appropriate KCL concentration BC buffer, and then re-suspended in 50 ul-100 ul elution buffer, containing 0.2 mg/ml Flag-peptide (Kodak) in BC 100 buffer, and incubated at 4° C. with rotation for one hour. The released FT-complex was then recuperated by centrifugal separation through a filter cartridge retaining the M2-agarose. Larger scale purifications were scaled for 50 ml-250 ml, using a maximum of 100 ul M2-agarose/250 ml and the same concentration of Flag-peptide. Complexes were then snap-frozen in liquid nitrogen.

Sequencing of P-TEFb and DSIF components. Complexes from the equivalent of 200 ml of nuclear extract were fractionated on a preparative gel, and specific bands identified in comparison to the control pCIN4 cell line complex by coomassie blue staining. Specific bands from the coomassie blue stained gel were cut into gel slices and submitted for in-gel digestion and sequence analysis. Peptide sequences were scanned against nr, EST and monthly updates using NCBI BLAST.

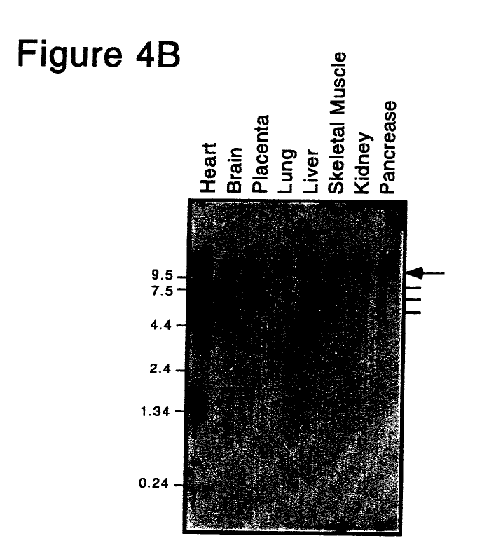

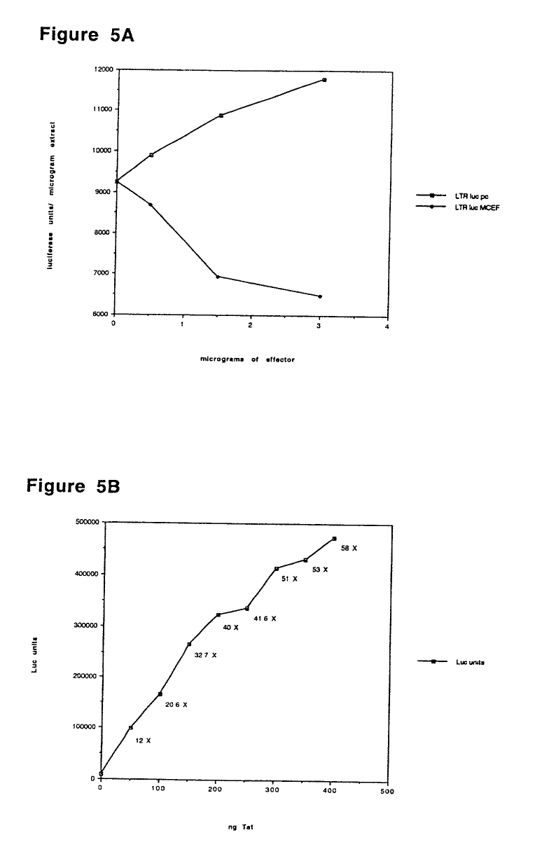

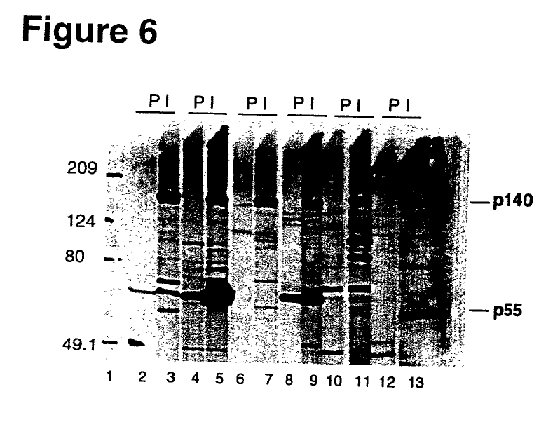

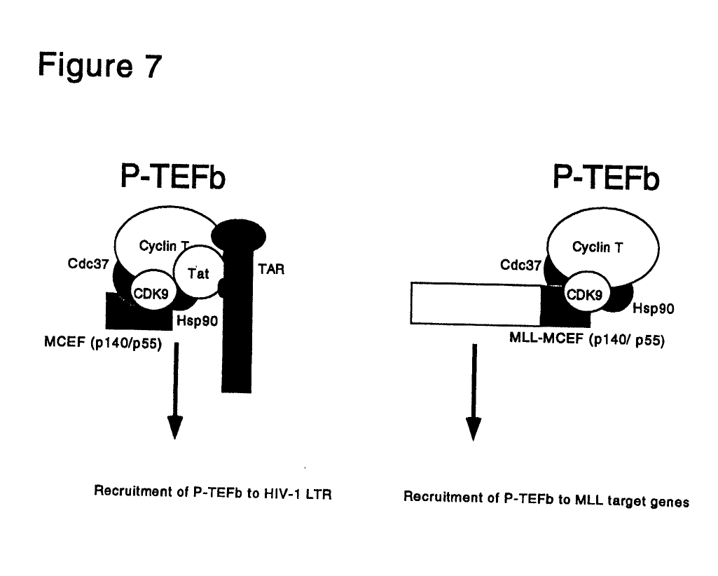

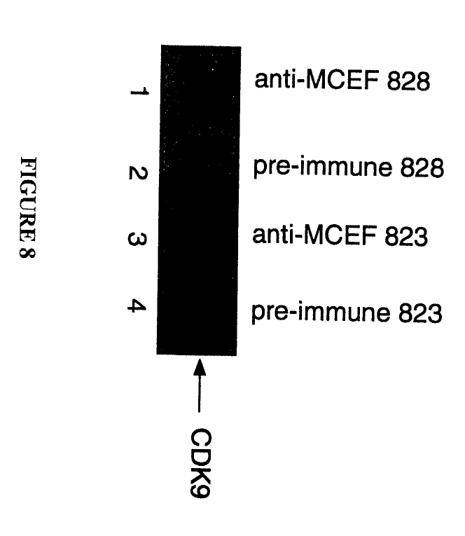

Cloning MCEF cDNA. EST clones were identified from GenBank sequence accession numbers matching the novel peptide sequence (FIG. 4A; a.a. position 110-121), and the corresponding ATCC clones ordered. The 874 bp and 819 bp sequences of ATCC 800100 and ATCC 823352 were determined using the −21M13 Forward and M13 Reverse primers and found to lack both an initiation codon and a stop codon. 5′- and 3′-RACE on ATCC 800100 were performed using HeLa-S3 cDNA by touchdown PCR (Clontech). Nested reaction primers used for 5′-RACE were AP-1 (Clontech) with MEGSP-1 (FIG. 4A; complement of underlined sequence nucleotides position 595-569), followed by AP-2 (Clontech) with MEGSP-2 (FIG. 4A; complement of underlined sequence nucleotide position, 557-529). For nested 3′-RACE primers used were AP-1 with GSP-3 (FIG. 4A; underlined sequence 402-422), followed by AP-2 (Clontech) with GSP-4 (FIG. 4A; underlined sequence 432-457). Amplicons were cloned into pT-Adv vector (Clontech). Clones were sequenced with the −21M13 Forward, the M13 Reverse primers and the appropriate GSP.

M13 forward primer: 5′d(CGCCAGGGTTTTCCCAGTCACGAC)-3′(SEQ ID NO:12)

M13 reverse primer: 5′-d(GGAAACAGCTATGACCATG)-3′(SEQ ID NO:13) The pTAdv-MCEF-5′-RACE-2.1 clone contained the first base indicated in FIG. 4A, and thus the ATG start codon. The longest pTAdv-MCEF-3′-RACE-8.10 clone terminated without a stop codon at position 1,941 followed by 20 A bases. A unique Bsu36I site shared by the 2.1 and 8.10 RACE clones was used to generate the partial clone pMCEF(1-1,914)-5, lacking a termination codon. In order to obtain the missing 3′-end, a Lambda ZAPII foetal brain cDNA library was screened (Stratagene) as per the manufacturers recommendations with a radiolabeled 570 bp probe from a BglII site at position 395 to a NcoI site at position 965 (FIG. 4A), encompassing the region identified by the peptide sequencing of the original P-TEFb novel EST band. After two rounds of screening, single plaques were picked and the pBluescript-MCEF-14-2A-1 phagemid was found to contain the furthest 3′-sequences but lacked 340 bases from the 5′-end when compared to pMCEF(1-1,914)-5. The final full length pBluescript-MCEF-FL-(1-5,858) was assembled using a unique NheI site of the pBluescript-MCEF-14-2A-1 and the pMCEF(1-1,914)-5.

Characterization of MCEF. Chromosomal location for MCEF was determined using BLAST. Alignments and phylogenetic trees with AF4/FMR2 family members are generated using ClustalW and cladistic analysis respectively(MacVector 6.5). Human Multiple Tissue Northern blot (Clonetech) were hybridized with the 570 bp probe used for the screening (see above) as per the manufacturers recommendations. For making rMCEF proteins, the sequences corresponding to the amino acid positions of MCEF were amplified with primers designed to generate a 5′-NdeI site and a 3′-BamHI site, and cloned into the NdeI and BamHI sites of pet-6HIS-11 d (Novagen). Respective clones were expressed in BL21 cells under IPTG induction and the best expressing clones used to generate excess of 1.5 mg of rMCEF proteins, nickel column purified and gel excised for injection into rabbits (COVANCE, standard protocol). For ectopic expression of MCEF in transient transfections, a of pcDNA constructs were generated. The pSV-Tat construct drove the expression of Tat exon 1 from the RSV promoter. The CDK9 cDNAs were obtained from Kathy Jones (Salk Institute, Calif.) [4]. The CDK9M was made by standard site directed mutagenesis from the CDK9 cDNA, and was a gift from Hua Xiao (Roeder Lab, Rockefeller University). The GST-CTD was made by standard cloning and expression techniques from the human Pol II CTD repeats, and was a gift, along with purified Pol II, from Hua Xiao (Roeder Lab, RU). The DSIFp160 cDNA was from Handa [26]. All of the cDNA derived clones used in this work (CDK9, DSIFp160, CTD) obtained from other investigators, can be readily made, by designing primers for amplification from their respective Genbank sequences, and cloning a respective amplification product from any human tissue. The pMCE-pcDNA-MCEF series of clones were generated by direct cloning of an amplicon encompassing the respective amino acids of MCEF, with an added 5′-XhoI site and a 3′-BamHI site, into the XhoI and BamHI sites of pcDNA3.1-(Invitrogen). The pGL2-HIV-1-LTR-luc reporter drove the expression of luciferase from the HIV-1 LTR. For transfections Jurkat E6-1 cells (ATCC) were grown and maintained in RPMI 1640 (JRH), supplemented with 10% BCS, 10 ml/L Pens. Strep. (as above). The standard assay contained 1.25 million cells transfected, using superfect (Qiagen) as per the manufacturers instructions, with 200 ng of pSV-Tat, 1 μg of PGL2-HIV-1-LTR-Luc and the indicated amounts of effector plasmid constructs. The dual luciferase assay system (Promega) was used to monitor reporter gene activities in a luminometer.

Preparation of MCEF or rMCEF substantially free of other human proteins

This can be achieved by sub-cloning the cDNA of MCEF into baculovirus pVL Vector 1392 and expressing it in Spodoptera Frugiterda SF21 cells. An alternative procedure, which produces small amounts of pure MCEF, is to immunoprecipitate it from a disassociated P-TEFb complex. FLAG-tagged MCEF can be similarly purified using anti-FLAG antibody.

CDC37,CDK9, Cyclin T1, Cyclin T2A, Cyclin T2B, HSP90α, and HSP90β

Each of these proteins can be generated by standard recombinant techniques in which the cDNA for the protein is integrated in expressible form (e.g. with a promoter) into a vector or plasmid, which is then used to transfect insect cells or other cells. The cDNA can be isolated by amplification from human cells based on a PCR reaction using a probe based on the known base sequence of the respective mRNA. Each of these proteins can also be isolated from human cells.

Epitope-tagged cell lines

The CDK9/CDK9M and DSIF p160 cDNAs were amplified by PCR using primer pairs creating a 5′-NdeI site and a 3′-BglII site. Pairs were: MEK9NdeI Forward: 5′-CGGAATTCCATATGGCAAAGCAGT ACGACTCG-3′; MEK9BglII Reverse: 5′-GGAAGATCTTCCTCAGAAGACGGGCTCT TTGAG-3′: MEp160NdeI Forward: 5′-CGGGAATTCCATATGTCGGACAGCGAGGAC AGC-3′; MEP160BglII Reverse: 5′-GGAAGATCTTCAGGCTTC-3′. A f:linker was annealed from the primer pair: MEf:EcoRI Upper: 5′-AATTCATGGACTACAAAGACGATGACGATAAACAT-3′; MEf:NdeI Lower: 5′-TATGTTTATCGTCATCGTCTTTGTAGTCCAT-3′ generating a EcoRI-f:-NdeI linker. The f:linkers and respectively digested PCR products were ligated in 3-way reactions into the EcoRI and BamHI sites of pCIN4 (pIRESneo, Clontech) to generate the constructs. Adherent HeLa-S cells (ATCC# CCL-2.2) were grown in DMEM (JRH) supplemented with 10% BCS (JRH) and 10 ml/L Penicillin-Streptomycin (Sigma # P-0906) and maintained in standard 37° C. CO₂ incubators. Hela-S3 cells were transfected using LipofectAMINE (GibcoBRL) per the manufacturers suggested protocol. Clones were selected with Geneticin (GibcoBRL) and screened for expression of the f:proteins by western blot with ANTI-FLAG M2 Monoclonal primary antibody (Sigma). Stocks for the newly created stable cell lines were stored in liquid nitrogen.

Immunopurification of P-TEFb and DSIF

Cells were grown to high density in a closed CO₂ system and in DMEM (JRH) containing 5% FBS. Nuclear and cytoplasmic extracts were prepared by the method of Dignam (Dignam et al., 1983). Extracts were either frozen directly in liquid nitrogen or pre-dialyzed to 100 mM KCl. Extracts were rotated at 4° C. for 1 hour in BC buffer (Dignam et al., 1983) containing the desired concentration of KCl with 0.1% NP-40 and M2-agarose. The respective M2-agarose-bound complexes were purified by centrifugation followed by 3 washes at the same KCL concentration. Complexes were eluted with 200 ng/ml FLAG peptide in BC 100 buffer and snap-frozen in liquid nitrogen. Complexes were fractionated on SDS-PAGE and either silver stained (KODAK), Coomassie blue stained or transferred to nitrocellulose membranes for western blotting. Complexes were confirmed to be P-TEFb and DSIF enzymatically by a standard kinase assay (Xiao et al., 2000) and by peptide sequencing. Anti-CDK9/PITALRE and cyclin T1 antibody was purchased from Santa Cruz. Anti-nucleolin antibody has been described previously (Parada and Roeder, 1999). Antibodies against HSP90a, HSP90b, HSP70 and CDC37 were purchased from Stressgene.

MCEF cloning and nucleic acid characterization

Complexes from the equivalent of 200 ml of nuclear extract were fractionated on a preparative SDS-PAGE gel and specific bands identified in comparison to the control pCIN4 cell line by Coomassie blue staining. Specific bands were cut into gel slices, in-gel digested and sequenced. Peptide sequences were compared to reported sequences using NCBI BLAST at LANL. EST clones were identified from genbank sequence accession numbers matching the novel peptide sequence and the corresponding ATCC clones were ordered. The 874 bp and 819 bp sequences of ATCC 800100 and ATCC 823352 were determined using the −21M13 Forward and M13 Reverse primers and found to lack both an initiation codon and a stop codon. 5′- and 3′-RACE on ATCC 800100 were performed using HeLa-S3 cDNA by touchdown PCR (Clontech). Nested reaction primers used for 5′-RACE were AP-1 (Clontech) with MEGSP-1 (nucleic acid position 595-569), followed by AP-2 (Clontech) with MEGSP-2 (557-529). Nested 3′-RACE primer pairs were AP-1 with GSP-3 (372-393), followed by AP-2 with GSP-4 (402-457). Amplicons were cloned into pT-Adv vector (Clontech). Clones were sequenced with the −21M13 Forward, the M13 Reverse and the appropriate GSP. The pTAdv-MCEF-5′-RACE-2.1 clone contained the ATG start codon. The longest pTAdv-MCEF-3′-RACE-8.10 clone terminated without a stop codon at position 1,941 followed by 20 A bases. A unique Bsu36I site shared by the 2.1 and 8.10 RACE clones was used to generate the partial clone pMCEF(1-1,914)-5, which lacks a termination codon. In order to obtain the missing 3′-end, a Lambda ZAPII fetal brain cDNA library was screened (Stratagene) per the manufacturers recommendations with a radiolabeled 570 bp probe from a BglII site at position 395 to a NcoI site at position 841-846, encompassing the region identified by the peptide sequencing of the original novel EST band. After two rounds of screening, single plaques were picked and the pBluescript-MCEF-14-2A-1 phagemeid was found to contain the furthest 3′-sequences but lacked 340 bases from the 5′-end when compared to pMCEF(1-1,914)-5. The final full length pBluescript-MCEF-FL-(1-5,858) was assembled using a unique NheI site of the pBluescript-MCEF-14-2A-1 and the pMCEF(1-1,914)-5 (GenBank 3088991). Chromosomal location for MCEF was determined using BLAST. Alignments and phylogenetic trees with AF4/FMR2 family members were generated using ClustalW and cladistic analysis respectively (MacVector 6.5). Human Multiple Tissue Northern blot (Clonetech) were hybridized with the 570 bp probe used for the screening (see above) as per the manufacturers recommendations.

Antibodies, western blots and immunoprecipitations

For making rMCEF proteins, the sequences corresponding to the amino acid positions of MCEF were amplified with primers designed to generate a 5′-NdeI site and a 3′-BamHI site, and cloned into the NdeI and BamHI sites of pet-6HIS-11d (Novagen). Respective clones were expressed in BL21 cells under IPTG induction and the best expressing clones used to generate excess of 1.5 mg of rMCEF proteins, nickel column purified and gel excised for injection into rabbits (COVANCE, standard protocol).

Viral replication and luciferase reporter assays

Cyclin T1 and CDK9 cDNAs were obtained from Dr. Kathy Jones (Salk Institute, Calif.). The CDK9M was made by standard site directed mutagenesis from the CDK9 cDNA and contains the D167N mutation (Zhou et al., 1998). The DSIF p160 cDNA was from Dr. Handa (Wada et al., 1998). The pcDNA-MCEF-SENSE construct was generated by direct cloning of an amplicon encompassing the respective amino acids of MCEF, with an added 5′-XhoI site and a 3′-BamHI site, into the XhoI and BamHI sites of pcDNA3.1-(Invitrogen). The ANTISENSE construct was made by sub-cloning into pcDNA+(Invitrogen). The pfrLuc construct drives the expression of luciferase from a minimal synthetic TATA-box containing promoter with 5 Gal4 DNA binding sites upstream of the TATA box (Stratagene). The pfrLuc2 reporter contains an enhancer cloned upstream of the 5 Gal4 DNA binding sites in pfrLuc, and was made by cloning the enhancer from the vector pEGFP-C2 (Clontech) into the HindII-PstI sites in pfrLuc using standard techniques. Transient transfections for luciferase assays were performed using superfect (Qiagen) and assayed in a luminometer by standard methods (Promega).

Transfection of HeLa cells was performed by using FuGene6 transfection reagent (Boehringer Mannheim) as described previously (Naghavi et al., 2001; Naghavi et al., 1999). A human growth hormone (HGH)-expressing construct (as an internal control for transfection efficiency) was included with the pNL4-3-infectious clone. Cell culture supernatants were collected at 24 hours and analysed in HGH ELISA (Boehringer Mannheim). To monitor the p24^{gag} production in transfections with pNL4-3-derived plasmids, cell culture supernatants were analyzed in a p24^{gag} antigen capture HIVAG-1 ELISA (Abbott, North Chicago, Ill.) according to the manufacturers instructions.

CDC37, CDK9, Cyclin T1, Cyclin T2A, Cyclin T2B, HSP90α, and HSP90β Sequences from GenBank

The following sequence information on these proteins was obtained from GenBank.

The notation “PROVISIONAL Ref#Seq” indicates that the GenBank record had the annothation “This is a provisional reference sequence record that has not yet been subject to human review. The final curated reference sequence record may be somewhat different from this one.”

(1) Homo sapiens CDC37

LOCUS NM_{—}007065 1559 bp mRNA

VERSION NM_{—}007065.1 GI:5901921

REFERENCE 1 (bases 1 to 1559)

AUTHORS Stepanova, L., Leng, X., Parker, S. B. and Harper, J. W.

JOURNAL Genes Dev. 10 (12), 1491-1502 (1996)

REFERENCE 2 (bases 1 to 1559)

AUTHORS Dai K, Kobayashi R and Beach D.

JOURNAL J. Biol. Chem. 271 (36), 22030-22034 (1996)

COMMENT REFSEQ: This reference sequence was derived from U43077.1.

PROVISIONAL Ref#Seq.

CDC37 mRNA/cDNA sequence

AAGGAAAGATGGTGGACTACAGCGTGTGGGACCACATTGAGGTGTCTGATGATGAAGACGAGACGCACCCCAACATCG
ACACGGCCAGTCTCTTCCGCTGGCGGCATCAGGCCCGGGTGGAACGCATGGAGCAGTTCCAGAAGGAGAAGGAGGAAC
TGGACAGGGGCTGCCGCGAGTGCAAGCGCAAGGTGGCCGAGTGCCAGAGGAAACTGAAGGAGCTGGAGGTGGCCGAGG
GCGGCAAGGCAGAGCTGGAGCGCCTGCAGGCCGAGGCACAGCAGCTGCGCAAGGAGGAGCGGAGCTGGGAGCAGAAGC
TGGAGGAGATGCGCAAGAAGGAGAAGAGCATGCCCTGGAACGTGGACACGCTCAGCAAAGACGGCTTCAGCAAGAGCA
TGGTAAATACCAAGCCCGAGAAGACGGAGGAGGACTCAGAGGAGGTGAGGGAGCAGAAACACAAGACCTTCGTGGAAA
AATACGAGAAACAGATCAAGCACTTTGGCATGCTTCGCCGCTGGGATGACAGCCAAAAGTACCTGTCAGACAACGTCC
ACCTGGTGTGCGAGGAGACAGCCAATTACCTGGTCATTTGGTGCATTGACCTAGAGGTGGAGGAGAAATGTGCACTCA
TGGAGCAGGTGGCCCACCAGACAATCGTCATGCAATTTATCCTGGAGCTGGCCAAGAGCCTAAAGGTGGACCCCCGGG
CCTGCTTCCGGCAGTTCTTCACTAAGATTAAGACAGCCGATCGCCAGTACATGGAGGGCTTCAACGACGAGCTGGAAG
CCTTCAAGGAGCGTGTGCGGGGCCGTGCCAAGCTGCGCATCGAGAAGGCCATGAAGGAGTACGAGGAGGAGGAGCGCA
AGAAGCGGCTCGGCCCCGGCGGCCTGGACCCCGTCGAGGTCTACGAGTCCCTCCCTGAGGAACTCCAGAAGTGCTTCG
ATGTGAAGGACGTGCAGATGCTGCAGGACGCCATCAGCAAGATGGACCCCACCGACGCAAAGTACCACATGCAGCGCT
GCATTGACTCTGGCCTCTGGGTCCCCAACTCTAAGGCCAGCGAGGCCAAGGAGGGAGAGGAGGCAGGTCCTGGGGACC
CATTACTGGAAGCTGTTCCCAAGACGGGCGATGAGAAGGATGTCAGTGTGTGACCTGCCCCAGCTACCACCGCCACCT
GCTTCCAGCCCCTATGTGCCCCTTTTCAGAAAACAGATAGATGCCATCTCGCCCGCTCCTGACTTCCTCTACTTGCGC
TGCTCGGCCCAGCCTGGGGGCCCGCCCAGCCCTCCCTGGCCTCTCCACTGTCTCCACTCTCCAGCGCCCATTCAAGTC
TCTGCTTTGAGTCAAGGGGCTTCACTGCCTGCAGCCCCCCATCAGCATTATGCCAAAGGCCGGGGGTCCGGAAGGGCA
GAGGTCACCAGGCTGGTCTACCAGGTAGTTGGGGAGGGTCCCCAGCCAAGGGGCCGGCTCTCGTCACTGGGCTCTGTT
TTCACTGTTCGTCTGCTGTCTGTGTCTTCTATTTGGCAAACAGCAATGATCTTCCAATAAAAGATTTCAGATGCTCA,

which is SEQ ID NO: 14.

CDC37 protein sequence

MVDYSVWDHIEVSDDEDETHPNIDTASLFRWRHQARVERMEQFQKEKEELDRGCRECKRKVAECQRKLKE
LEVAEGGKAELERLQAEAQQLRKEERSWEQKLEEMRKKEKSMPWNVDTLSKDGFSKSMVNTKPEKTEEDS
EEVREQKHKTFVEKYEKQIKHFGMLRRWDDSQKYLSDNVHLVCEETANYLVIWCIDLEVEEKCALMEQVA
HQTIVMQFILELAKSLKVDPRACFRQFFTKIKTADRQYMEGFNDELEAFKERVRGRAKLRIEKAMKEYEE
EERKKRLGPGGLDPVEVYESLPEELQKCFDVKDVQMLQDAISKMDPTDAKYHMQRCIDSGLWVPNSKASE
AKEGEEAGPGDPLLEAVPKTGDEKDVSV,

which is SEQ ID NO: 15.

(2) Homo sapiens cyclin-dependent kinase 9 (CDK9)

LOCUS NM_{—}001261 1461 bp mRNA

VERSION NM_{—}001261.1 GI:4502746

REFERENCE 1 (bases 1 to 1461)

AUTHORS Grana, X., De Luca, A., Sang, N., Fu, Y., Claudio, P. P., Rosenblatt, J., Morgan, D. O. and Giordano, A.

JOURNAL Proc. Natl. Acad. Sci. U.S.A. 91, 3834-3838 (1994)

COMMENT REFSEQ: This reference sequence was derived from L25676.

PROVISIONAL RefSeq.

CDK9 mRNA/cDNA sequence

CGGGACCCGAGCAGGAGCGGCGGCACGAGCAGCTGGGGGCGGCGGCGGCGCGTTGGAGGCGGCCATGGCA
AAGCAGTACGACTCGGTGGAGTGCCCTTTTTGTGATGAAGTTTCCAAATACGAGAAGCTCGCCAAGATCG
GCCAAGGCACCTTCGGGGAGGTGTTCAAGGCCAGGCACCGCAAGACCGGCCAGAAGGTGGCTCTGAAGAA
GGTGCTGATGGAAAACGAGAAGGAGGGGTTCCCCATTACAGCCTTGCGGGAGATCAAGATCCTTCAGCTT
CTAAAACACGAGAATGTGGTCAACTTGATTGAGATTTGTCGAACCAAAGCTTCCCCCTATAACCGCTGCA
AGGGTAGTATATACCTGGTGTTCGACTTCTGCGAGCATGACCTTGCTGGGCTGTTGAGCAATGTTTTGGT
CAAGTTCACGCTGTCTGAGATCAAGAGGGTGATGCAGATGCTGCTTAACGGCCTCTACTACATCCACAGA
AACAAGATCCTGCATAGGGACATGAAGGCTGCTAATGTGCTTATCACTCGTGATGGGGTCCTGAAGCTGG
CAGACTTTGGGCTGGCCCGGGCCTTCAGCCTGGCCAAGAACAGCCAGCCCAACCGCTACACCAACCGTGT
GGTGACACTCTGGTACCGGCCCCCGGAGCTGTTGCTCGGGGAGCGGGACTACGGCCCCCCCATTGACCTG
TGGGGTGCTGGGTGCATCATGGCAGAGATGTGGACCCGCAGCCCCATCATGCAGGGCAACACGGAGCAGC
ACCAACTCGCCCTCATCAGTCAGCTCTGCGGCTCCATCACCCCTGAGGTGTGGCCAAACGTGGACAACTA
TGAGCTGTACGAAAAGCTGGAGCTGGTCAAGGGCCAGAAGCGGAAGGTGAAGGACAGGCTGAAGGCCTAT
GTGCGTGACCCATACGCACTGGACCTCATCGACAAGCTGCTGGTGCTGGACCCTGCCCAGCGCATCGACA
GCGATGACGCCCTCAACCACGACTTCTTCTGGTCCGACCCCATGCCCTCCGACCTCAAGGGCATGCTCTC
CACCCACCTGACGTCCATGTTCGAGTACTTGGCACCACCGCGCCGGAAGGGCAGCCAGATCACCCAGCAG
TCCACCAACCAGAGTCGCAATCCCGCCACCACCAACCAGACGGAGTTTGAGCGCGTCTTCTGAGGGCCGG
CGCTTGCCACTAGGGCTCTTGTGTTTTTTTTCTTCTGCTATGTGACTTGCATCGTGGAGACAGGGCATTT
GAGTTTATATCTCTCATGCATATTTTATTTAATCCCCACCCTGGGCTCTGGGAGCAGCCCGCTGAGTGGA
CTGGAGTGGAGCATTGGCTGAGAGACCAGGAGGGCACTGGAGCTGTCTTGTCCTTGCTGGTTTTCTGGAT
GGTTCCCAGAGGGTTTCCATGGGGTAGGAGGATGGGCTCGCCCACCAGTGACTTTTTCCCG,

which is SEQ ID NO: 16.

CDK9 protein sequence

MAKQYDSVECPFCDEVSKYEKLAKIGQGTFGEVFKARHRKTGQKVALKKVLMENEKEGFPITALREIKIL
QLLKHENVVNLIEICRTKASPYNRCKGSIYLVFDFCEHDLAGLLSNVLVKFTLSEIKRVMQMLLNGLYYI
HRNKILHRDMKAANVLITRDGVLKLADFGLARAFSLAKNSQPNRYTNRVVTLWYRPPELLLGERDYGPPI
DLWGAGCIMAEMWTRSPIMQGNTEQHQLALISQLCGSITPEVWPNVDNYELYEKLELVKGQKRKVKDRLK
AYVRDPYALDLIDKLLVLDPAQRIDSDDALNHDFFWSDPMPSDLKGMLSTHLTSMFEYLAPPRRKGSQIT
QQSTNQSRNPATTNQTEFERVF,

which is SEQ ID NO: 17.

(3) Homo sapiens cyclin T1 (CCNT1):

LOCUS NM_{—}001240 2360 bp mRNA

VERSION NM_{—}001240.1 GI:4502626

REFERENCE 1 (bases 1 to 2360)

AUTHORS Peng, J., Zhu, Y., Milton, J. T. and Price, D. H.

JOURNAL Genes Dev. 12 (5), 755-762 (1998)

REFERENCE 2 (bases 1 to 2360)

AUTHORS Peng, J. M., Zhu, Y., Milton, J. T. and Price, D. H.

Direct Submission

JOURNAL Submitted (Feb. 17, 1998) Biochemistry, University of Iowa, 51 Newton Road, Iowa City, Iowa 52242, USA

COMMENT REFSEQ: This reference sequence was derived from AF048730.

PROVISIONAL RefSeq:

Cyclin T1 mRNA/cDNA sequence

```
GGAAGTGCCTGCAACCTTCGCCGCTGCCTTCTGGTTGAAGCACTATGGAGGGAGAGAGGAAGAACAACAA
              CAAACGGTGGTATTTCACTCGAGAACAGCTGGAAAATAGCCCATCCCGTCGTTTTGGCGTGGACCCAGAT
              AAAGAACTTTCTTATCGCCAGCAGGCGGCCAATCTGCTTCAGGACATGGGGCAGCGTCTTAACGTCTCAC
              AATTGACTATCAACACTGCTATAGTATACATGCATCGATTCTACATGATTCAGTCCTTCACACGGTTCCC
              TGGAAATTCTGTGGCTCCAGCAGCCTTGTTTCTAGCAGCTAAAGTGGAGGAGCAGCCCAAAAAATTGGAA
              CATGTCATCAAGGTAGCACATACTTGTCTCCATCCTCAGGAATCCCTTCCTGATACTAGAAGTGAGGCTT
              ATTTGCAACAAGTTCAAGATCTGGTCATTTTAGAAAGCATAATTTTGCAGACTTTAGGCTTTGAACTAAC
              AATTGATCACCCACATACTCATGTAGTAAAGTGCACTCAACTTGTTCGAGCAAGCAAGGACTTAGCACAG
              ACTTCTTACTTCATGGCAACCAACAGCCTGCATTTGACCACATTTAGCCTGCAGTACACACCTCCTGTGG
              TGGCCTGTGTCTGCATTCACCTGGCTTGCAAGTGGTCCAATTGGGAGATCCCAGTCTCAACTGACGGGAA
              GCACTGGTGGGAGTATGTTGACGCCACTGTGACCTTGGAACTTTTAGATGAACTGACACATGAGTTTCTA
              CAGATTTTGGAGAAAACTCCCAACAGGCTCAAACGCATTTGGAATTGGAGGGCATGCGAGGCTGCCAAGA
              AAACAAAAGCAGATGACCGAGGAACAGATGAAAAGACTTCAGAGCAGACAATCCTCAATATGATTTCCCA
              GAGCTCTTCAGACACAACCATTGCAGGTTTAATGAGCATGTCAACTTCTACCACAAGTGCAGTGCCTTCC
              CTGCCAGTCTCCGAAGAGTCATCCAGCAACTTAACCAGTGTGGAGATGTTGCCGGGCAAGCGTTGGCTGT
              CCTCCCAACCTTCTTTCAAACTAGAACCTACTCAGGGTCATCGGACTAGTGAGAATTTAGCACTTACAGG
AGTTGATCATTCCTTACCACAGGATGGTTCAAATGCATTTATTTCCCAGAAGCAGAATAGTAAGAGTGTG
              CCATCAGCTAAAGTGTCACTGAAAGAATACCGCGCGAAGCATGCAGAAGAATTGGCTGCCCAGAAGAGGC
              AACTGGAGAACATGGAAGCCAATGTGAAGTCACAATATGCATATGCTGCCCAGAATCTCCTTTCTCATCA
              TGATAGCCATTCTTCAGTCATTCTAAAAATGCCCATAGAGGGTTCAGAAAACCCCGAGCGGCCTTTTCTG
              GAAAAGGCTGACAAAACAGCTCTCAAAATGAGAATCCCAGTGGCAGGTGGAGATAAAGCTGCGTCTTCAA
              AACCAGAGGAGATAAAAATGCGCATAAAAGTCCATGCTGCAGCTGATAAGCACAATTCTGTAGAGGACAG
              TGTTACAAAGAGCCGAGAGCACAAAGAAAAGCACAAGACTCACCCATCTAATCATCATCATCATCATAAT
              CACCACTCACACAAGCACTCTCATTCCCAACTTCCAGTTGGTACTGGGAACAAACGTCCTGGTGATCCAA
              AACATAGTAGCCAGACAAGCAACTTAGCACATAAAACCTATAGCTTGTCTAGTTCTTTTTCCTCTTCCAG
              TTCTACTCGTAAAAGGGGACCCTCTGAAGAGACTGGAGGGGCTGTGTTTGATCATCCAGCCAAGATTGCC
              AAGAGTACTAAATCCTCTTCCCTAAATTTCTCCTTCCCTTCACTTCCTACAATGGGTCAGATGCCTGGGC
              ATAGCTCAGACACAAGTGGCCTTTCCTTTTCACAGCCCAGCTGTAAAACTCGTGTCCCTCATTCGAAACT
              GGATAAAGGGCCCACTGGGGCCAATGGTCACAACACGACCCAGACAATAGACTATCAAGACACTGTGAAT
              ATGCTTCACTCCCTGCTCAGTGCCCAGGGTGTTCAGCCCACTCAGCCCACTGCATTTGAATTTGTTCGTC
              CTTATAGTGACTATCTGAATCCTCGGTCTGGTGGAATCTCCTCGAGATCTGGCAATACAGACAAACCCCG
              GCCACCACCTCTGCCATCAGAACCTCCTCCACCACTTCCACCCCTTCCTAAGTAAAAAAAGAAAAAGAAG
              AGGAGAAAAAAACTTCTTTAAAAAAACACATAATTTTTCTTTTTTTTTTGGGGAAAAAAAAATTTTTTTT
AAAATTTTTTCCCCAAGGGACGGGGGAAAATTTTATTTTTAAAATTTTTT, which is SEQ ID NO: 18.
```

Cyclin T1 protein sequence

```
MEGERKNNNKRWYFTREQLENSPSRRFGVDPDKELSYRQQAANLLQDMGQRLNVSQLTINTAIVYMHRFY
              MIQSFTRFPGNSVAPAALFLAAKVEEQPKKLEHVIKVAHTCLHPQESLPDTRSEAYLQQVQDLVILESII
              LQTLGFELTIDHPHTHVVKCTQLVRASKDLAQTSYFMATNSLHLTTFSLQYTPPVVACVCIHLACKWSNW
              EIPVSTDGKHWWEYVDATVTLELLDELTHEFLQILEKTPNRLKRIWNWRACEAAKKTKADDRGTDEKTSE
              QTILNMISQSSSDTTIAGLMSMSTSTTSAVPSLPVSEESSSNLTSVEMLPGKRWLSSQPSFKLEPTQGHR
              TSENLALTGVDHSLPQDGSNAFISQKQNSKSVPSAKVSLKEYRAKHAEELAAQKRQLENMEANVKSQYAY
              AAQNLLSHHDSHSSVILKMPIEGSENPERPFLEKADKTALKMRIPVAGGDKAASSKPEEIKMRIKVHAAA
              DKHNSVEDSVTKSREHKEKHKTHPSNHHHHHNHHSHKHSHSQLPVGTGNKRPGDPKHSSQTSNLAHKTYS
              LSSSFSSSSSTRKRGPSEETGGAVFDHPAKIAKSTKSSSLNFSFPSLPTMGQMPGHSSDTSGLSFSQPSC
              KTRVPHSKLDKGPTGANGHNTTQTIDYQDTVNMLHSLLSAQGVQPTQPTAFEFVRPYSDYLNPRSGGISS
RSGNTDKPRPPPLPSEPPPPLPPLPK, which is SEQ ID NO: 19.
```

(4) Homo sapiens cyclin T2a

LOCUS AF048731 4499 bp mRNA

VERSION AF048731.1 GI:2981197

SOURCE human.

REFERENCE 1 (bases 1 to 4499)

AUTHORS Peng, J., Zhu, Y., Milton, J. T. and Price, D. H.

JOURNAL Genes Dev. 12 (5), 755-762 (1998)

REFERENCE 2 (bases 1 to 4499)

AUTHORS Peng, J. M., Zhu, Y., Milton, J. T. and Price, D. H.

Direct Submission.

JOURNAL Submitted (Feb. 17, 1998) Biochemistry, University of Iowa, 51 Newton Road, Iowa City, Iowa 52242, USA

Cyclin T2A mRNA/cDNA sequence

```
TGAATGAAGGAGCGGGCGGAGGAGGAATTGTCATGGCGTCGGGCCGTGGAGCTTCTTCTCGCTGGTTCTT
              TACTCGGGAACAGCTGGAGAACACGCCGAGCCGCCGCTGCGGAGTGGAGGCGGATAAAGAGCTCTCGTGC
              CGCCAGCAGGCGGCCAACCTCATCCAGGAGATGGGACAGCGTCTCAATGTCTCTCAGCTTACAATAAACA
              CTGCGATTGTTTATATGCACAGGTTTTATATGCACCATTCTTTCACCAAATTCAACAAAAATATAATATC
              GTCTACTGCATTATTTTTGGCTGCAAAAGTGGAAGAACAGGCTCGAAAACTTGAACATGTTATCAAAGTA
              GCACATGCTTGTCTTCATCCTCTAGAGCCACTGCTGGATACTAAATGTGATGCTTACCTTCAACAGACTC
              AAGAACTGGTTATACTTGAAACCATAATGCTACAAACTCTAGGTTTTGAGATCACCATTGAACACCCACA
              CACAGATGTGGTGAAATGTACCCAGTTAGTAAGAGCAAGCAAGGATTTGGCACAGACATCCTATTTCATG
GCTACCAACAGTCTGCATCTTACAACCTTCTGTCTTCAGTACAAACCAACAGTGATAGCATGTGTATGCA
              TTCATTTGGCTTGCAAATGGTCCAATTGGGAGATCCCTGTATCAACTGATGGAAAGCATTGGTGGGAATA
              TGTGGATCCTACAGTTACTCTAGAATTATTAGATGAGCTAACACATGAGTTTCTACAAATATTGGAGAAA
              ACGCCTAATAGGTTGAAGAAGATTCGAAACTGGAGGGCTAATCAGGCAGCTAGGAAACCAAAAGTAGATG
              GACAGGTATCAGAGACACCACTTCTTGGTTCATCTTTGGTCCAGAATTCCATTTTAGTAGATAGTGTCAC
              TGGTGTGCCTACAAACCCAAGTTTTCAGAAACCATCTACATCAGCATTCCCTGCGCCAGTACCTCTAAAT
              TCAGGAAATATTTCTGTTCAAGACAGCCATACATCTGATAATTTGTCAATGCTAGCAACAGGAATGCCAA
              GTACTTCATACGGTTTATCATCACACCAGGAATGGCCTCAACATCAAGACTCAGCAAGGACAGAACAGCT
              ATATTCACAGAAACAGGAGACATCTTTGTCTGGTAGCCAGTACAACATCAACTTCCAGCAGGGACCTTCT
              ATATCACTGCATTCAGGATTACATCACAGACCTGACAAAATTTCAGATCATTCTTCTGTTAAGCAAGAAT
              ATACTCATAAAGCAGGGAGCAGTAAACACCATGGGCCAATTTCCACTACTCCAGGAATAATTCCTCAGAA
              AATGTCTTTAGATAAATATAGAGAAAAGCGTAAACTAGAAACTCTTGATCTCGATGTAAGGGATCATTAT
              ATAGCTGCCCAGGTAGAACAGCAGCACAAACAAGGGCAGTCACAGGCAGCCAGCAGCAGTTCTGTTACTT
              CTCCCATTAAAATGAAAATACCTATCGCAAATACTGAAAAATACATGGCAGATAAAAAGGAAAAGAGTGG
              GTCACTGAAATTACGGATTCCAATACCACCCACTGATAAAAGCGCCAGTAAAGAAGAACTGAAAATGAAA
              ATAAAAGTTTCTTCTTCAGAAAGACACAGCTCTTCTGATGAAGGCAGTGGGAAAAGCAAACATTCAAGCC
              CACATATTAGCAGAGACCATAAGGAGAAGCACAAGGAGCATCCTTCAAGCCGCCACCACACCAGCAGCCA
              CAAGCATTCCCACTCGCATAGTGGCAGCAGCAGCGGTGGCAGTAAACACAGTGCCGACGGAATACCACCC
              ACTGTTCTGAGGAGTCCTGTTGGCCTGAGCAGTGATGGCATTTCCTCTAGCTCCAGCTCTTCAAGGAAGA
              GGCTGCATGTCAATGATGCATCTCACAACCACCACTCCAAAATGAGCAAAAGTTCCAAAAGTTCAGGTGG
              GCTACGGACATCTCAGCACCCTCGTGAAACTGGACAAGAAGCCAGTGGAGACCAACGGTCCTGATGCCAA
              TCACGAGTACAGTACAAGCAGCCAGCATATGGACTACAAAGACACATTCGACATGCTGGACTCACTGTTA
AGTGCCCAAGGAATGAACATGTAATAATTTGTTTAGGTCAATTTTTCCTTTACTTTTTTAATTTAAAAAT
              TGTTAGAATGGAAAAATTCCTTCTGATCTAGCAGTGGTAACCCCTGCTGTTGCTGCCACTGCTTCAATAT
              TTGTAAGTGCTACTTTATTCTTCATTCTGAAAAGAAGAGATTATAGTAAACAAGTCTTTATCTCCACATA
              TGATAGTGTTATAAATACTGTAAAGGCATGGAAGGTGCAAAACTCAGTATTTCTACAATTGCAGCTAAGA
              ACATTAGGATGAATGGCTGGCTGCTTCTAGGAATATAAGATGCCTCAAGCATTCATTATTTATGATTTGA
              ATACTGTAGCTATTTTTTGTTGCTTGGCTTTTGAATGAGTGTAAATTGTTTTCTTTTGTGTATTTATACT
              TGTATGTATGATTTGCATGTTTCAATGATAAAGGGATAAAACAGTATACTGACAACTGTTTACAAGAAAG
              TGGAGAAAATGTACTACATTTTGTATGTTTAGATATTACCGTAAATACTCAGGATTGGAGCTGCTTGTAA
              GTATAACAATATACAGAATACTTTATTTTATCTTGTCAGAGTTCCATCACTATCTAAAACAAAGGTGCAA
              TTTTTTATGTTAACCTTAAATCTAGCCCTTACTGGAAGCCACTGATAGGGACATTCACTACCAGATGTGT
              GCAGTGCAGCAGATGGTCATATAACACTGTGAGGCACTGAATTTTGCCTTCAGAGGTTCTGACCAGATTG
              GCTGCTGAAATAGCCCCTAACTTTCTGAAGGCTTGAAGAGGAAAAAATAAAGTTTACATACTCTTGATGG
              AAGTGCATTTAAATGTTTGTTGGCTTGTTGCAGTTCTATGAAACAGAGCTGTTAATAATGGTTATGTGGA
              TTACTGTGATTTGAAAACTAAATTCACAATAACTTACCTAGTAGAGATTTAGTGAGTTGTTTCCTTTAAA
              GAATTTTACACTACATATTTTAATAGTAAACAGGGTCACTTTCCTTTAGCATTCAGAATGACACCATATT
              CTTAAATATACTCCTTCCCTGAAGCGTGTTTGTGTGTGATGCCATATTTCTTTTTCAGGTAAATGTAGTC
              TTCCTTATAAAAATGAAATTAAACCTATGCTCTCAATTCTTTTATATTCTAACAATAAATAAAAAAGAAA
AGATTACTGACTGTGCATTGTACCTGTATTTATAGTTTATGGTTATCAGAAGCTCTGTAAGAAAGAAAAG
              GTCAGCTCCCAGGCAAACCAGTAGTGGAGGTTTTACATTTGTTTGCACATCTCAGTATATTTCTGTTGAG
              GTAAAGTTTGCACAGTCATCTGACTTCTGATCAAGCATTAGATTTTAACTTGTTTAGATTTTGTCTTAAA
              CACCAGTAATATGGCTCTTGTTTATCAGCTAATCTTGAATTTATTCTGTGGTAAATCTTTTGAGTTGCTG
              AGTATATTTGAGATTGATTGGATTCAACCTCTTGTTGAACTGAAAACTTAATTTTTTCTCTGTATTTTTG
              TTACAAAGCCACTGATACGTGCACAATTGTAATTAAGTATGTTGCAGTTGTAAATATTAGAGTTTAATCT
              CATGCTCTACCTTTATTTAGCAATTACCTAATTTGCCAGTAGCTTTATAATTTTTAAAGATAATTGTTCA
              TTATTTTGTCAATGTTATTTGAACTTGGGGTACTTAGGAGCCTCTTTGTAGGGACTGTGCCTAGGTAGCA
              TGTCCTAACATTTGTTCTGGTCTTGCATAACTTCAGTATCTTTGTCATTATATGTAACTTTGTTGCTCTG
              TATGGCATAATATTGTATCCATAAACATGGTAATTTTGATACAGTTATACTTTTACAGTGGTACATAATC
              CAAGGACTAGTATAGAATTAAGCTGAGTGCAAGATGAGGGAGGGAAGGGCTTTCTTGGTAATTTAGATGT
              GAAACCTCTACAGAGCTATCATGTAAAAACTACATGAGGTGGTTGTGCTACTGTATAATTGGGGGTGATA
              ATACCAGGAATTTTAATAAGATTTTGTAAAGAATATCCAGAAAAGTAGTGAACTTATTTTCAGTAGGCAT
              AGAAAACAATGTGAATATTTAAGGTCTGTGACTATAGTTAAACTTCACTAAGAATTTGCAGAATTGTTTT
              GAGATGTGTGAATAAAGGTAATTTTATTGAATCTTCATTGGTGCTAATGTTGGACAGTTAAAAAGATAGC
              TAGTGTATATTGTTATGGGTCAGTACTTATTAGTACTTCCAAAATTGAATTTGAAATGCTATGTATTCAC
              TTTTCACTCTGTAAATGTAATTCTTTACAATGACTTTATTTATTAAAGGGCAGCCAGTTGTCATTTGTAA
AAAAAAAAAAAAAAAAAAA, which is SEQ ID NO: 20.
```

Cyclin T2A protein sequence

```
MASGRGASSRWFFTREQLENTPSRRCGVEADKELSCRQQAANLIQEMGQRLNVSQLTINTAIVYMHRFYM
HHSFTKFNKNIISSTALFLAAKVEEQARKLEHVIKVAHACLHPLEPLLDTKCDAYLQQTQELVILETIML
              QTLGFEITIEHPHTDVVKCTQLVRASKDLAQTSYFMATNSLHLTTFCLQYKPTVIACVCIHLACKWSNWE
              IPVSTDGKHWWEYVDPTVTLELLDELTHEFLQILEKTPNRLKKIRNWRANQAARKPKVDGQVSETPLLGS
              SLVQNSILVDSVTGVPTNPSFQKPSTSAFPAPVPLNSGNISVQDSHTSDNLSMLATGMPSTSYGLSSHQE
              WPQHQDSARTEQLYSQKQETSLSGSQYNINFQQGPSISLHSGLHHRPDKISDHSSVKQEYTHKAGSSKHH
              GPISTTPGIIPQKMSLDKYREKRKLETLDLDVRDHYIAAQVEQQHKQGQSQAASSSSVTSPIKMKIPIAN
              TEKYMADKKEKSGSLKLRIPIPPTDKSASKEELKMKIKVSSSERHSSSDEGSGKSKHSSPHISRDHKEKH
              KEHPSSRHHTSSHKHSHSHSGSSSGGSKHSADGIPPTVLRSPVGLSSDGISSSSSSSRKRLHVNDASHNH
HSKMSKSSKSSGGLRTSQHPRETGQEASGDQRS, which is SEQ ID NO: 21.
```

(5) Homo sapiens cyclin T2b:

LOCUS AF048732 2193 bp mRNA

VERSION AF048732.1 GI:2981199

REFERENCE 1 (bases 1 to 2193)

JOURNAL Genes Dev. 12 (5), 755-762 (1998)

REFERENCE 2 (bases 1 to 2193)

AUTHORS Peng, J. M., Zhu, Y., Milton, J. T. and Price, D. H.

Direct Submission

JOURNAL Submitted (Feb. 17, 1998) Biochemistry, University of Iowa, 51 Newton Road, Iowa City, Iowa 52242, USA

Cyclin T2b mRNA/cDNA sequence

```
ATGGCGTCGGGCCGTGGAGCTTCTTCTCGCTGGTTCTTTACTCGGGAACAGCTGGAGAACACGCCGAGCC
GCCGCTGCGGAGTGGAGGCGGATAAAGAGCTCTCGTGCCGCCAGCAGGCGGCCAACCTCATCCAGGAGAT
              GGGACAGCGTCTCAATGTCTCTCAGCTTACAATAAACACTGCGATTGTTTATATGCACAGGTTTTATATG
              CACCATTCTTTCACCAAATTCAACAAAAATATAATATCGTCTACTGCATTATTTTTGGCTGCAAAAGTGG
              AAGAACAGGCTCGAAAACTTGAACATGTTATCAAAGTAGCACATGCTTGTCTTCATCCTCTAGAGCCACT
              GCTGGATACTAAATGTGATGCTTACCTTCAACAGACTCAAGAACTGGTTATACTTGAAACCATAATGCTA
              CAAACTCTAGGTTTTGAGATCACCATTGAACACCCACACACAGATGTGGTGAAATGTACCCAGTTAGTAA
              GAGCAAGCAAGGATTTGGCACAGACATCCTATTTCATGGCTACCAACAGTCTGCATCTTACAACCTTCTG
              TCTTCAGTACAAACCAACAGTGATAGCATGTGTATGCATTCATTTGGCTTGCAAATGGTCCAATTGGGAG
              ATCCCTGTATCAACTGATGGAAAGCATTGGTGGGAATATGTGGATCCTACAGTTACTCTAGAATTATTAG
              ATGAGCTAACACATGAGTTTCTACAAATATTGGAGAAAACGCCTAATAGGTTGAAGAAGATTCGAAACTG
              GAGGGCTAATCAGGCAGCTAGGAAACCAAAAGTAGATGGACAGGTATCAGAGACACCACTTCTTGGTTCA
              TCTTTGGTCCAGAATTCCATTTTAGTAGATAGTGTCACTGGTGTGCCTACAAACCCAAGTTTTCAGAAAC
              CATCTACATCAGCATTCCCTGCGCCAGTACCTCTAAATTCAGGAAATATTTCTGTTCAAGACAGCCATAC
              ATCTGATAATTTGTCAATGCTAGCAACAGGAATGCCAAGTACTTCATACGGTTTATCATCACACCAGGAA
              TGGCCTCAACATCAAGACTCAGCAAGGACAGAACAGCTATATTCACAGAAACAGGAGACATCTTTGTCTG
              GTAGCCAGTACAACATCAACTTCCAGCAGGGACCTTCTATATCACTGCATTCAGGATTACATCACAGACC
              TGACAAAATTTCAGATCATTCTTCTGTTAAGCAGGAATATACTCATAAAGCAGGGAGCAGTAAACACCAT
              GGGCCAATTTCCACTACTCCAGGAATAATTCCTCAGAAAATGTCTTTAGATAAATATAGAGAAAAGcGTA
AACTAGAAACTCTTGATCTCGATGTAAGGGATCATTATATAGCTGCCCAGGTAGAACAGCAGCACAAACA
              AGGGCAGTCACAGGCAGCCAGCAGCAGTTCTGTTACTTCTCCCATTAAAATGAAAATACCTATCGCAAAT
              ACTGAAAAATACATGGCAGATAAAAAGGAAAAGAGTGGGTCACTGAAATTACGGATTCCAATACCACCCA
              CTGATAAAAGCGCCAGTAAAGAAGAACTGAAAATGAAAATAAAAGTTTCTTCTTCAGAAAGACACAGCTC
              TTCTGATGAAGGCAGTGGGAAAAGCAAACATTCAAGCCCACATATTAGCAGAGACCATAAGGAGAAGCAC
              AAGGAGCATCCTTCAAGCCGCCACCACACCAGCAGCCACAAGCATTCCCACTCGCATAGTGGCAGCAGCA
              GCGGTGGCAGTAAACACAGTGCCGACGGAATACCACCCACTGTTCTGAGGAGTCCTGTTGGCCTGAGCAG
TGATGGCATTTCCTCTAGCTCCAGCTCTTCAAGGAAGAGGCTGCATGTCAATGATGCATCTCACAACCAC
              CACTCCAAAATGAGCAAAAGTTCCAAAAGTTCAGGTAGTTCATCTAGTTCTTCCTCCTCTGTTAAGCAGT
              ATATATCCTCTCACAACTCTGTTTTTAACCATCCCTTACCCCCTCCTCCCCCTGTCACATACCAGGTGGG
              CTACGGACATCTCTGCACCCTCGTGAAACTGGACAAGAAGCCAGTGGAGACCAACGGTCCTGATGCCAAT
              CACGAGTACAGTACAAGCAGCCAGCATATGGACTACAAAGACACATTCGACATGCTGGACTCACTGTTAA
GTGCCCAAGGAATGAACATGTAA, which is SEQ ID NO: 22.
```

Cyclin T2b protein sequence

```
MASGRGASSRWFFTREQLENTPSRRCGVEADKELSCRQQAANLIQEMGQRLNVSQLTINTAIVYMHRFYM
HHSFTKFNKNIISSTALFLAAKVEEQARKLEHVIKVAHACLHPLEPLLDTKCDAYLQQTQELVILETIML
              QTLGFEITIEHPHTDVVKCTQLVRASKDLAQTSYFMATNSLHLTTFCLQYKPTVIACVCIHLACKWSNWE
              IPVSTDGKHWWEYVDPTVTLELLDELTHEFLQILEKTPNRLKKIRNWRANQAARKPKVDGQVSETPLLGS
              SLVQNSILVDSVTGVPTNPSFQKPSTSAFPAPVPLNSGNISVQDSHTSDNLSMLATGMPSTSYGLSSHQE
              WPQHQDSARTEQLYSQKQETSLSGSQYNINFQQGPSISLHSGLHHRPDKISDHSSVKQEYTHKAGSSKHH
              GPISTTPGIIPQKMSLDKYREKRKLETLDLDVRDHYIAAQVEQQHKQGQSQAASSSSVTSPIKMKIPIAN
              TEKYMADKKEKSGSLKLRIPIPPTDKSASKEELKMKIKVSSSERHSSSDEGSGKSKHSSPHISRDHKEKH
              KEHPSSRHHTSSHKHSHSHSGSSSGGSKHSADGIPPTVLRSPVGLSSDGISSSSSSSRKRLHVNDASHNH
              HSKMSKSSKSSGSSSSSSSSVKQYISSHNSVFNHPLPPPPPVTYQVGYGHLCTLVKLDKKPVETNGPDAN
HEYSTSSQHMDYKDTFDMLDSLLSAQGMNM, which is SEQ ID NO: 23.
```

(6) HSP90 alpha

LOCUS HSHSP90R 2912 bp mRNA

ACCESSION X15183

VERSION X15183.1 GI:32487

SOURCE human.

REFERENCE 1 (bases 1 to 2912)

AUTHORS Yokoyama, K.

Direct Submission.

JOURNAL Submitted (May 2, 1989) Yokoyama K., Gene Bank Riken, The Institute of Physical and Chemical Research, Tsukuba Life Science Centre, Koyadai 3-1-1 Tukuba, Ibaraki 305, Japan

REFERENCE 2 (bases 1 to 2906)

AUTHORS Yamazaki, M., Akaogi, K., Miwa, T., Imai, T., Soeda, E. and Yokoyama, K.

JOURNAL Nucleic Acids Res. 17 (17), 7108 (1989)

REFERENCE 3 (bases 1 to 2912)

AUTHORS Aligue, R., Akhavan-Niak, H. and Russell, P.

JOURNAL EMBO J. 13 (24), 6099-6106 (1994)

```
HSP90 alpha, mRNA/cDNA
CAGTTGCTTCAGCGTCCCGGTGTTGCTGTGCCGTTGGTCCTGTGCGGTCACTTAGCCAAGATGCCTGAGG
              AAACCCAGACCCAAGACCAACCGATGGAGGAGGAGGAGGTTGAGACGTTCGCCTTTCAGGCAGAAATTGC
              CCAGTTGATGTCATTGATCATCAATACTTTCTACTCGAACAAAGAGATCTTTCTGAGAGAGCTCATTTCA
              AATTCATCAGATGCATTGGACAAAATCCGGTATGAAACTTTGACAGATCCCAGTAAATTAGACTCTGGGA
              AAGAGCTGCATATTAACCTTATACCGAACAAACAAGATCGAACTCTCACTATTGTGGATACTGGAATTGG
AATGACCAAGGCTGACTTGATCAATAACCTTGGTACTATCGCCAAGTCTGGGACCAAAGCGTTCATGGAA
GCTTTGCAGGCTGGTGCAGATATCTCTATGATTGGCCAGTTCGGTGTTGGTTTTTATTCTGCTTATTTGG
TTGCTGAGAAAGTAACTGTGATCACCAAACATAACGATGATGAGCAGTACGCTTGGGAGTCCTCAGCAGG
GGGATCATTCACAGTGAGGACAGACACAGGTGAACCTATGGGTCGTGGAACAAAAGTTATCCTACACCTG
AAAGAAGACCAAACTGAGTACTTGGAGGAACGAAGAATAAAGGAGATTGTGAAGAAACATTCTCAGTTTA
TTGGATATCCCATTACTCTTTTTGTGGAGAAGGAACGTGATAAAGAAGTAAGCGATGATGAGGCTGAAGA
AAAGGAAGACAAAGAAGAAGAAAAAGAAAAAGAAGAGAAAGAGTCGGAAGACAAACCTGAAATTGAAGAT
GTTGGTTCTGATGAGGAAGAAGAAAAGAAGGATGGTGACAAGAAGAAGAAGAAGAAGATTAAGGAAAAGT
ACATCGATCAAGAAGAGCTCAACAAAAGAAAGCCCATCTGGACCAGAAATCCCGACGATATTACTAATGA
GGAGTACGGAGAATTCTATAAGAGCTTGACCAATGACTGGGAAGATCACTTGGCAGTGAAGCATTTTTCA
GTTGAAGGACAGTTGGAATTCAGAGCCCTTCTATTTGTCCCACGACGTGCTCCTTTTGATCTGTTTGAAA
ACAGAAAGAAAAAGAACAATATCAAATTGTATGTACGCAGAGTTTTCATCATGGATAACTGTGAGGAGCT
AATCCCTGAATATCTGAACTTCATTAGAGGGGTGGTAGACTCGGAGGATCTCCCTCTAAACATATCCCGT
GAGATGTTGCAACAAAGCAAAATTTTGAAAGTTATCAGGAAGAATTTGGTCAAAAAATGCTTAGAACTCT
TTACTGAACTGGCGGAAGATAAAGAGAACTACAAGAAATTCTATGAGCAGTTCTCTAAAAACATAAAGCT
TGGAATACACGAAGACTCTCAAAATCGGAAGAAGCTTTCAGAGCTGTTAAGGTACTACACATCTGCCTCT
GGTGATGAGATGGTTTCTCTCAAGGACTACTGCACCAGAATGAAGGAGAACCAGAAACATATCTATTATA
TCACAGGTGAGACCAAGGACCAGGTAGCTAACTCAGCCTTTGTGGAACGTCTTCGGAAACATGGCTTAGA
AGTGATCTATATGATTGAGCCCATTGATGAGTACTGTGTCCAACAGCTGAAGGAATTTGAGGGGAAGACT
TTAGTGTCAGTCACCAAAGAAGGCCTGGAACTTCCAGAGGATGAAGAAGAGAAAAAGAAGCAGGAAGAGA
AAAAAACAAAGTTTGAGAACCTCTGCAAAATCATGAAAGACATATTGGAGAAAAAAGTTGAAAAGGTGGT
TGTGTCAAACCGATGGTAGACATCTCCATGCTGTATTGTCACAAGCACATATGGCTGGACAGCAAACATG
GAGAGAATCATGAAAGCTCAAGCCCTAAGAGACAACTCAACAATGGGTTACATGGCAGCAAAGAAACACC
TGGAGATAAACCCTGACCATTCCATTATTGAGACCTTAAGGCAAAAGGCAGAGGCTGATAAGAACGACAA
GTCTGTGAAGGATCTGGTCATCTTGCTTTATGAAACTGCGCTCCTGTCTTCTGGCTTCAGTCTGGAAGAT
CCCCAGACACATGCTAACAGGATCTACAGGATGATCAAACTTGGTCTGGGTATTGATGAAGATGACCCTA
CTGCTGATGATACCAGTGCTGCTGTAACTGAAGAAATGCCACCCCTTGAAGGAGATGACGACACATCACG
CATGGAAGAAGTAGACTAATCTCTGGCTGAGGGATGACTTACCTGTTCAGTACTCTACAATTCCTCTGAT
AATATATTTTCAAGGATGTTTTTCTTTATTTTTGTTAATATTAAAAAGTCTGTATGGCATGACAACTACT
TTAAGGGGAAGATAAGATTTCTGTCTACTAAGTGATGCTGTGATACCTTAGGCACTAAAGCAGAGCTAGT
AATGCTTTTTGAGTTTCATGTTGGTTCTTTCACAGATGGGGTAACGTGCACTGTAAGACGTATGTAACAT
GATGTTAACTTTGTGTGGTCTAAAGTGTTTAGCTGTCAAGCCGGATGCCTAAGTAGACCAAATCTTGTTA
TTGAAGTGTTCTGAGCTGTATCTTGATGTTTAGAAAAGTATTCGTTACATCTTTGTAGGATCTACTTTTG
AACTTTTCATTCCCTGTAGTTGACAATTCTGCATGTACTAGTCCTCTAGAAATAGGTTAAACTGAAGCAA
CTTGATGGAAGGATCTCTCCACAGGGCTTGTTTTCCAAAGAAAAGTATTGTTTGGAGGAGCAAAGTTAAA
AGCCTACCTAAGCATATCGTAAAGCTGTTCAAATACTCGAGCCCAGTCTTGTGGATGGAAATGTAGTGCT
CGAGTCACATTCTGCTTAAAGTTGTAACAAATACAGATGAGT, which is SEQ ID NO:24.
```

```
HSP 90 alpha, protein sequence
MPEETQTQDQPMEEEEVETFAFQAEIAQLMSLIINTFYSNKEIPFRELISNSSDALDKIRYETLTDPSKL
              DSGKELHINLIPNKQDRTLTIVDTGIGMTKADLINNLGTIAKSGTKAFMEANQAGADISMIGQFGVGFYS
              AYLVAEKVTVITKHNDDEQYAWESSAGGSFTVRTDTGEPMGRGTKVILHLKEDQTEYLEERRIKEIVKKH
              SQFIGYPITLFVEKERDKEVSDDEAEEKEDKEEEKEKEEKESEDKPEIEDVGSDEEEEKKDGDKKKKKKI
              KEKYIDQEELNKTKPIWTRNPDDITNEEYGEFYKSLTNDWEDHLAVKHFSVEGQLEFRALLFVPRRAPFD
              LFENRKKKNNIKLYVRRVFIMDNCEELIPEYLNFIRGVVDSEDLPLNISREMLQQSKILKVIRKNLVKKC
              LELFTELAEDKENYKKFYEQFSKNIKLGIDEDSQNRKKLSELLRYYTSASGDEMVSLKDYCTRMKENQKH
              IYYITGETKDQVANSAFVERLRKHGLEVIYMIEPIDEYCVQQLKEFEGKTLVSVTKEGLELPEDEEEKKK
              QEEKKTKFENLCKIMKDILEKKVEKVVVSNRLVTSPCCIVTSTYGWTANMERIMKAQALRDNSTMGYMAA
              KKHLEINPDHSIIETLRQKAEADKNDKSVKDLVILLYETALLSSGFSLEDPQTHANRIYRMIKLGLGIDE
DDPTADDTSAAVTEEMPPLEGDDDTSRMEEVD, which is SEQ ID NO:25.
```

(7) Homo sapiens HSP90β

LOCUS NM_{—}007355 2459 bp mRNA

ACCESSION NM_{—}007355

VERSION NM_{—}007355.1 GI:6680306

SOURCE human.

REFERENCE 1 (bases 1 to 2459)

AUTHORS Rebbe, N. F., Ware, J., Bertina, R. M., Modrich, P. and Stafford, D. W.

JOURNAL Gene 53, 235-245 (1987)

MEDLINE 87277414

REFERENCE 2 (bases 1 to 2459)

AUTHORS Rebbe N F, Hickman W S, Ley T J, Stafford D W and Hickman S.

JOURNAL J. Biol. Chem. 264 (25), 15006-15011 (1989)

REFERENCE 3 (bases 1 to 2459)

AUTHORS Takahashi I, Tanuma R, Hirata M and Hashimoto K.

JOURNAL Mamm. Genome 5 (2), 121-122 (1994)

COMMENT REFSEQ: This reference sequence was derived from M16660.1.

PROVISIONAL RefSeq.

```
HSP90 beta, mRNA/cDNA sequence
ATGCCTGAGGAAGTGCACCATGGAGAGGAGGAGGTGGAGACTTTTGCCTTTCAGGCAGAAATTGCCCAAC
TCATGTCCCTCATCATCAATACCTTCTATTCCAACAAGGAGATTTTCCTTCGGGAQTTGATCTCTAATGC
TTCTGATGCCTTGGACAAGATTCGCTATGAGAGCCTGACAGACCCTTCGAAGTTGGACAGTGGTAAAGAG
CTGAAAATTGACATCATCCCCAACCCTCAGGAACGTACCCTGACTTTGGTAGACACAGGCATTGGCATGA
CCAAAGCTGATCTCATAAATAATTTGGGAACCATTGCCAAGTCTGGTACTAAAGCATTCATGGAGGCTCT
TCAGGCTGGTGCAGACATCTCCATGATTGGGCAGTTTGGTGTTGGCTTTTATTCTGCCTACTTGGTGGCA
GAGAAAGTGGTTGTGATCAGAAAGCACAACGATGATGAACAGTATGCTTGGGAGTCTTCTGCTCGAGGTT
CCTTCACTGTGCGTGCTGACCATGGTGAGCCCATTGGCATGGGTACCAAAGTGATCCTCCATCTTAAAGA
AGATCAGACAGAGTACCTAGAAGAGAGGCGGGTCAAAGAAGTAGTGAAGAAGCATTCTCAGTTCATAGGC
TATCCCATCACCCTTTATTTGGAGAAGGAACGAGAGAAGGAAATTAGTGATGATGAGGCAGAGGAAGAGA
AAGGTGAGAAAGAAGAGGAAGATAAAGATGATGAAGAAAAGCCCAAGATCGAAGATGTGGGTTCAGATGA
GGAGGATGACAGCGGTAAGGATAAGAAGAAGAAAACTAAGAAGATCAAAGAGAAATACATTGATCAGGAA
GAACTAAACAAGACCAAGCCTATTTGGACCAGAAACCCTGATGACATCACCCAAGAGGAGTATGGAGAAT
  TCTACAAGACCCTCACTAATGACTCGGAAGACCACTTGGCAGTCAAGCACTTTTCTGTAGAAGGTCAGTT
              GGAATTCAGGGCATTGCTATTTATTCCTCGTCGGGCTCCCTTTGACCTTTTTGAGAACAAGAAGAAAAAG
              AACAACATCAAACTCTATGTCCGCCGTGTGTTCATCATGGACAGCTGTGATGAGTTGATACCAGAGTATC
              TCAATTTTATCCGTGGTGTGGTTGACTCTGAGGATCTGCCCCTGAACATCTCCCGAGAAATGCTCCAGCA
              GAGCAAAATCTTGAAAGTCATTCGCAAAAACATTGTTAAGAAGTGCCTTGAGCTCTTCTCTGAGCTGGCA
              GAAGACAAGGAGAATTACAAGAAATTCTATGAGGCATTCTCTAAAAATCTCAAGCTTGGAATCCACGAAG
              ACTCCACTAACCGCCGCCGCCTGTCTGAGCTGCTGCGCTATCATACCTCCCAGTCTGGAGATGAGATGAC
              ATCTCTGTCAGAGTATGTTTCTCGCATGAAGGAGACACAGAAGTCCATCTATTACATCACTGGTGAGAGC
              AAAGAGCAGGTGGCCAACTCAGCTTTTGTGGAGCGAGTGCGGAAACGGGGCTTCGAGGTGGTATATATGA
              CCGAGCCCATTGACGAGTACTGTGTGCAGCAGCTCAAGGAATTTGATGGGAAGAGCCTGGTCTCAGTTAC
              CAAGGAGGGTCTGGAGCTGCCTGAGGATGAGGAGGAGAAGAAGAAGATGGAAGAGAGCAAGGCAAAGTTT
              GAGAACCTCTGCAAGCTCATGAAAGAAATCTTAGATAAGAAGGTTGAGAAGGTGACAATCTCCAATAGAC
              TTGTGTCTTCACCTTGCTGCATTGTGACCAGCACCTACGGCTGGACAGCCAATATGGAGCGGATCATGAA
AGCCCAGGCACTTCGGGACAACTCCACCATGGGCTATATGATGGCCAAAAAGCACCTGGAGATCAACCT
              GACCACCCCATTGTGGAGACGCTGCGGCAGAAGGCTGAGGCCGACAAGAATGATAAGGCAGTTAAGGACC
              TGGTGGTGCTGCTGTTTGAAACCGCCCTGCTATCTTCTGGCTTTTCCCTTGAGGATCCCCAGACCCACTC
              CAACCGCATCTATCGCATGATCAAGCTAGGTCTAGGTATTGATGAAGATGAAGTGGCAGCAGAGGAACCC
              AATGCTGCAGTTCCTGATGAGATCCCCCCTCTCGAGGGCGATGAGGATGCGTCTCGCATGGAAGAAGTCG
              ATTAGGTTAGGAGTTCATAGTTGGAAAACTTGTGCCCTTGTATAGTGTCCCCATGGGCTCCCACTGCAGC
              CTCGAGTGCCCCTGTCCCACCTGGCTCCCCCTGCTGGTGTCTAGTGTTTTTTCCCTCTCCTGTCCTTGT
              GTTGAAGGCAGTAAACTAAGGGTGTCAAGCCCCATTCCCTCTCTACTCTTGACAGCAGGATTGGATGTTG
              TGTATTGTGGTTTATTTTATTTTCTTCATTTTGTTCTGAAATTAAAGTATGCAAAATAAAGAATATGCCG
TTTTTATAC, which is SEQ ID NO:26.
```

HSP90 beta, protein sequence
MPEEVHHGEEEVETFAFQAEIAQLMSLIINTFYSNKEIFLRELISNASDALDKRIYESLTDPSKLDSGKELKIDIIPNPQE
RTLTLVDTGIGMTKADLINNLGTIAKSGTKAFMEALQAGADISMIGQFGVGFYSAYLVAEKVVVIRKHNDDEQYAWESSAG
GSFTVRADHGEPIGMGTKVILHLKEDQTEYLEERRVKEVVKKHSQFIGYPITLYLEKEREKEISDDEAEEEKGEKEEEDKD
DEEKPKIEDVGSDEEDDSGKDKKKKTKKIKEKYIDQEELNKTKPIWTRNPDDITQEEYGEFYKSLTNDWEDHLAVKHFSVE
GQLEFRALLFIPRRAPFDLFENKKKKNNIKLYVRRVFIMDSCDELIPEYLNFIRGVVDSEDLPLNISREMLQQSKILKVIR
KNIVKKCLELFSELAEDKENYKKFYEAFSKNLKLGIHEDSTNRRRRLSELLRYHTSQSGDEMTSLSEYVSRMETQKSIYYI
TGESKEQVANSAFVERVRKRGFEVVYMTEPIDEYCVQQLKEFDGKSLVSVTKEGLELPEDEEEKKKMEESKAKFENLCKLM
KEILDKKVEKVTISNRLVSSPCCIVTSTYGWTANMERIMKAQALRDNSTMGYMMAKKHLEINPDHPIVETLRQKAEADKND
KAVKDLVVLLFETALLSSGFSLEDPQTHSNRIYRMIKLGLGIDEDEVAAEEPNAAVPDEIPPLEGDEDASRMEEVD,
which is SEQ ID NO:27.

Preferred base sequence for the TAR region

This base sequence is:
GGGTCTCTCTGGTTAGACCAGATCTGAGCCTGGGAGCTCTCTGGCTAACTAGGGAACCC which is SEQ ID NO:28.

### EXAMPLES

The following examples are intended to illustrate the invention, not limit it.

### Example 1

The ME-HeLa-S3-CIN4-FT-CDK9-7, ME-HeLa-S3-CIN4-FT-CDK9M-9 and ME-HeLa-S3-CIN4DSIFp160-8, cell lines express FT-CDK9, FT-CDK9M and FT-DSIFp160 respectively.

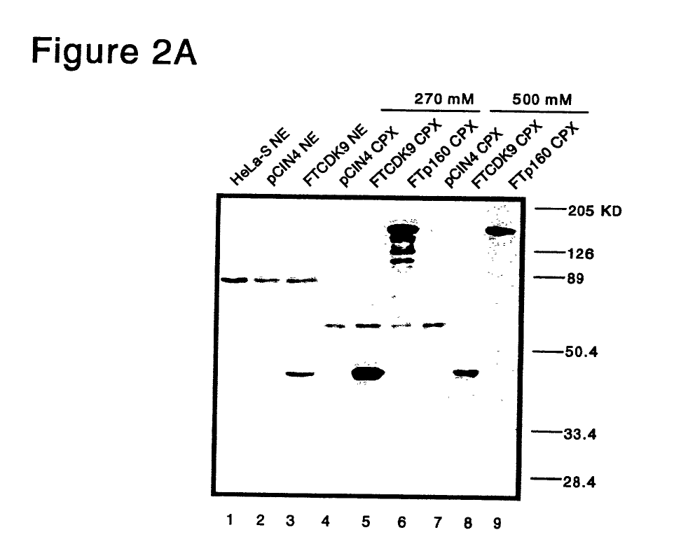

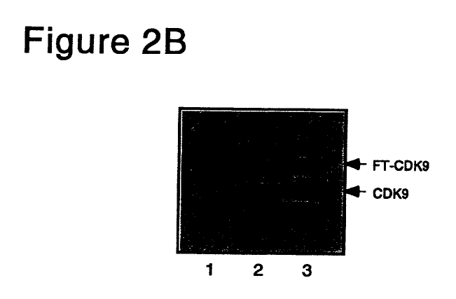

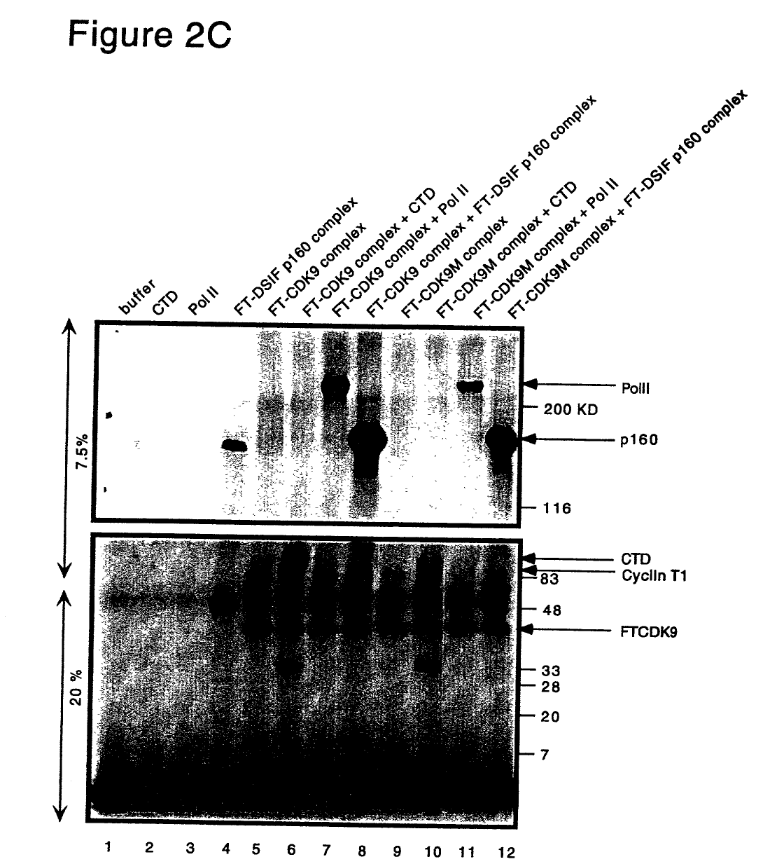

In order to address the question of what components are contained in P-TEFb, we opted for the strategy depicted in FIG. 1A. Fig. 1B shows some representative examples of whole cell extracts from some of the clones screened. The positive clones make FT-products circa 160 kD for the SPT5-8, or of circa 45 kD for the CDK9-7, CDK9M-9 (Fig. 1B, lanes 3, 5 and 6). The expected size for DSIFp160 is 160 kD [26], and for CDK9/CDK9M is 45 KD [25] [4]. We conclude here that we have made stable cell lines expressing DSIFp160, CDK9 or CDK9M FLAG-tagged proteins. The stable cell lines ME-Hela-S3-CIN4-FT-CDK9-7(FIG. 1B, lane 5, FIG. 2A lanes 3, 5, 8) ME-HeLa-S3-CIN4-FT-CDK9M-9 (FIG. 1B, lane 6) and ME-HeLa-S3-CIN4-DSIFp160-8, (FIG. 1B, lane 3; FIG. 2A, lanes 6, 9)that stably express FT-CDK9, FT-CDK9M and FT-DSIFp160 respectively and the control cell line ME-HeLa-S3-CIN4-1 (FIG. 2A, lanes 2, 4, 7), were selected for expansion and immunopurification of P-TEFb and DSIF.

### Example 2

An active P-TEFb complex contains the chaperone pair HSP-90-CDC37, Cyclin T1, T2A, T2B, CDK9 and the novel gene product MCEF

The immunopurified FT-CDK9, FT-CDK9M and FT-DSIFp160 were prepared at 100 mM, 270 mM, 350 mM and 500 mM KCl concentrations in BC buffers [28]. In nuclear extracts, a FT-antibody immunoreactive-band in the 80 kD range was non-specific (FIG. 2A. compare lanes 1, 2, 3 with lanes 4-9). In both the270 mM and 500 mM immunopurifications, the 80 kD non-specific immunoreactive band was absent, but at 270 mM a circa 70 kD non-specific immunoreactive band was present (lanes 4, 5, 6). At 500 mM KCl, this band was absent from both the FT-CDK9 and FT-DSIF-p160 preparations (lanes 8, 9). The 500 mM KCL FT-CDK9 immunopurification contained predominantly the FT-CDK9 protein (FIG. 2B, lane 1, top arrow, right hand side of figure). Because the immunopurified material appears to be more specific at higher salt concentrations (above and also FIG. 3), we used the 500 mM material to test for the ability of the FT-CDK9 immunopurification to phosphorylate the CTD of pol II (FIG. 2C). The FT-CDK9 immunopurification autophosphorylates specific components circa 45 kD and 87 kD, consistent with the molecular weights expected for Cyclin T1 and a FT-CDK9 (Lane 5) [25] [4]. Both CDK9 and Cyclin T1 are known to be autophosphorylated by CDK9 in P-TEFb [25] [4]. The FT-CDK9 immunopurification can also phosphorylate rGST-CTD of Pol II (Lane 6, CTD arrow) or Pol II itself (Lane 7, Pol II arrow). Interestingly, the FT-CDK9 immunopurification can heavily phosphorylate a band circa 160 kD in the FT-DSIFp160 preparation, likely to be FT-DSIFp160 (Lane 8). The FT-CDK9M immunopurification was significantly weaker in its ability to autophosphorylate the 87 kD band (Lane 9) or to phosphorylate the GST-CTD or Pol II (Lanes 10, 11) and was weaker in its ability to phosphorylate the 160 kD band of the FT-DSIFp160 immunopurification (Lane 12). These results implicate CDK9 as being the major active kinase in the 500 mM FT-CDK9 immunopreparation, consistent with our having immunopurified an enzymatically active P-TEFb.

In order to confirm that the immunopurified material is P-TEFb, and to unambiguously identify its components, we resolved the preparations on acrylamide gels and extracted the specific component bands for direct sequencing. As suggested by the western blots with FLAG-tagged antibody (above), SDS-PAGE silver staining shows a more specific group of bands immunopurifying using 500 mM KCl (FIG. 3). The specific bands could be immunopurified from both the nuclear or S-100 extracts (FIG. 3). The FT-CDK9 and FT-CDK9M preparations used in FIG. 2, showed equivalent bands, discarding the possibility that the decreased kinase activity (FIG. 2C) was due to a different group of bands associating with the FT-CDK9M versus the FT-CDK9 material (FIG. 3C). The results of peptide sequencing for the FT-CDK9 and FT-DSIFp160 500 mM KCL immunopurified components are indicated in FIGS. 3A and B. Surprisingly, the most prominent band in a stoicheometric ratio with FT-CDK9 was found to be HSP90. The occurrence of this band in conjunction with the occurrence of Cdc37, suggests a HSP90-Cdc37 chaperone pair for CDK9 (see discussion). As expected, and confirming that we have purified a bonafide P-TEFb complex, Cyclin T1, T2A, T2B were detected. Importantly, the peptide sequence VGPAPSTSQSQK (SEQ ID NO:1), matching an EST for a novel and uncharacterized cDNA was detected (FIG. 3A).

We conclude here that an active P-TEFb immunopurified from our ME-HeLa-S3-FT-CDK9-7 contains HSP90-Cdc37, Cyclin T1, T2A, T2B and a novel gene product, which we name MCEF.

### Example 3

A 5,858 bp cDNA clone was isolated for MCEF, which is a novel member of the AF4/FMR2 family of transcription activators, encoded on chromosome 5.

The cloning of MCEF through a combination of RACE and Lambda cDNA screening is described in materials and methods. The final clone encompassed 5,858 bp and a major ORF for a theoretical 715 aa protein, matching the 12 aa sequence (VGPAPSTSQSQK) (SEQ ID NO:1) was identified (FIG. 4A, aa position 110-121). A major 10 kb transcript was detected in heart, brain placenta, lung, liver, skeletal muscle, pancreas (FIG. 4B, arrow). The strongest expression appears to be in heart, where at least 3 minor transcripts of approximately 5, 7 and 8 kD appear to be expressed (FIG. 4B, position indicated by lines under arrow, right hand side). In addition the same result of a 10 kb transcript was detected in immune tissue from human spleen, lymph nodes, thymus, peripheral blood leukocyte, bone marrow and fetal liver, all known to be permissive tissues for HIV infection in vivo. Database BLAST searches indicated MCEF DNA, and the major MCEF ORF theoretical protein (MCEF protein), to be potential members of the AF4/FMR2 family of transcription activators. An alignment of the 4 sequences best matching the major MCEF DNA indicates significant homology exists around the mapped AF4 strong transactivation domain [29]. Phylogenetic analysis indicates that hMCEF, hAF4, hFMR2, hLAF, mLAF are an outgroup from the next nearest sequence in the Swiss-Protein database, hNF . This demonstrates that these sequences indeed form a distinct family set. Further database analysis identified the genomic sequences from which MCEF cDNA is derived as being on chromosome 5, near the Interleukin gene cluster, within Alu repeat sequences. Preliminary analysis indicates that MCEF mRNA is encoded by as many as 16 exons, spanning 50 kb.

We conclude here that the novel component of human P-TEFb is the gene product of MCEF, encoded on chromosome 5, from as many as 16 exons, near the Interleukin gene cluster.

### Example 4

Confirmation of MCEF involvement in P-TEFb

In order to further test if MCEF is truly a component of P-TEFb, we generated 3 different groups of antibodies to N-terninal portions of rMCEF (rMCEF 1-715, 1-530, 1-420), contained within MCEF by injecting recombinant proteins into rabbits and collecting sera (see Materials and Methods) (FIG. 6). These antibodies (sera) were used in western blots with nuclear extracts and are used with immunopurified material. As seen in FIG. 6, there are at least 2 bands, p140 and p55, of 140 kD and 55kD, in HeLa nuclear extracts.

As a further test to the involvement of MCEF in P-TEFb, we selected the HIV-1 LTR [30-32], a promoter well characterized for its requirement of P-TEFb in Tat transactivation (Reviewed in: [3]). FIG. 5B shows the system for Tat transactivation in vivo, to which MCEF-expressing constructs can be applied. The results obtained for a contro -without an MCEF construct are shown in FIG. 5B.

FIG. 5A shows an effect of the ectopically expressed MCEF on transcription directed from the HIV-1 LTR. This effect confirms that ectopic expression of MCEF can affect HIV-1 LTR transcription, in vivo, in T Cells. These same cells, we have shown, express both the p140 and p55 MCEF protein bands.

### Example 5

Affinity test for agents that bind to MCEF

For the in vitro binding assays one can make a histidine-tagged recombinant region of MCEF or rMCEF (e.g., one used for injection into rabbits in FIG. 6) followed by binding of the his-MCEF protein to a nickel column. Reagents suspected of binding to rMCEF (such as other recombinant proteins, or any antibody-reactive or otherwise detectable reagent) can be passed over the column. The column is then washed with excess buffer (such as, buffer BC100) and the bound reagent can be eluted with varying concentrations of salt. For in vivo binding, the well known yeast two hybrid system can be used with MCEF as bait. (obtainable from Clontech).

For testing for agents that bind to other PTEF-b components, the same approach is used as for MCEF, except that the component of interest, or its recombinant version, is substituted for MCEF or rMCEF. For example, for HSP-90 binding reagents or CDC37-binding reagents, the same approach is used as for MCEF.

### Example 6

Assay for anti-MCEF HSP90 CDC37 reagents of use against leukemia

One sets up a cell growth assay, where cells derived from tissues that have undergone a MCEF gene family translocation (such as MLL-AF4 fusions or MLL-chromosome 5 MCEF fusions) are grown in tissue culture. Reagents (preferably that have been by other means found to interact with MCEF, HSP90 or CDC37), are then tested for their ability to interfere with the growth of these same leukemia-derived cell.

### Example 7

Assay for anti-MCEF reagents or other anti-P-TEFb component reagents of use against leukemia

One sets-up a cell growth assay, where cells derived from ti ssues that have undergone a MCEF gene family translocation (such as MLL-AF4 fusions or MLL-chromosome 5 MCEF fusions) are grown in tissue culture. Reagents, for example, those that have been by other assays found to interact with MCEF, or other P-TEFb components, are then tested for their ability to interfere with the growth of these same leukemia-derived cells.

### Example 8

Assay for anti-MCEF reagents or other anti-P-TEFb component reagents of use against HIV

In order to search for reagents that may interfere with the involvement of MCEF or other PTEFb components in HIV transcription, one sets-up an in vivo transient transfection assay for Tat transactivation (as above, example 4) [30, 31], and uses known inhibitors for HSP90 (such as Geldanamycin) or selected reagents based on logical assumptions that their effects may interfere with P-TEFb activities or random screens for reagents by automated methods.

As another possibility, one sets up a replication assay for HIV (see below, example 7) and then tests the reagents found to bind (by in vitro or in vivo binding assays, see above, example 5) to MCEF, or other components of PTEF-b, or the reagents found to interfere with Tat transactivation (as above, this example) and tests them for their ability to interfere with HIV replication.

### Example 9

Effect of MCEF on HIV transcription or replication

For examining the effect of MCEF protein on HIV transcription we used the transient transfection system in Jurkat cells as described by Estable [30, 31] and mentioned above in Example 4. The MCEF expression constructs are then applied to the system shown in FIG. 5.

The human T-cell lymphoma line Jurkat or HeLa, can be used to assay the effects of rMCEF on HIV-1 replication. HIV and a plasmid expressing MCEF (such as any of the pcDNA series, see above in Materials and Methods) are co-transfected into the cell line, by standard techniques. Cell culture supernatants are then collected at various times posttransfection (0, 24, 48, 72, and 92h) and HIV-1 p24gag production are measured in cell culture supernatants in a p24gag antigen capture HIVAG-1 ELISA (Abbott, North Chicago, Ill.) according to the manufacturer instructions.

### Example 10

Administration of an MCEF-binding ragent or other P-TEFb-component binding reagent to a patient

Pharmaceutical preparations of anti-MCEF reagents or other P-TEFb-component binding reagent (or anti-HSP90 reagents or antiCDC37 reagents) would include pharmaceutically acceptable carriers, or other adjuvants as needed, and would be prepared in effective dosage ranges as needed. Liposomes and cationic lipids might also be used to deliver the reagents inhibitors inside cells.

Generally, the anti-MCEF reagents (or precursors thereof capable of being correctly processed in the host or the host's cells) of the invention may be formulated for intraarterial, intraperitoneal, intramuscular, subcutaneous, intravenous, oral, nasal, rectal, bucal, sublingual, pulmonary, topical, transdermal, or other routes of administration. Comprehended by the invention are pharmaceutical compositions comprising effective amounts of anti-MCEF reagents together with pharmaceutically acceptable diluents, preservatives, solubilizers, emulsifiers, adjuvants and/or carriers. Such compositions include, for example, aqueous diluents of various buffer content, incorporation of the material into particulate preparations of polymeric compounds such as polylactic acid, polyglycolic acid, etc. or into liposomes. The compositions may be prepared in liquid form, or may be in dried powder, such as lyophilized fonn.

Solid dosage forms include tablets, capsules, pills, troches or lozenges, cachets or pellets. Also, liposomal or proteinoid encapsulation may be used to formulate the present compositions (as, for example, proteinoid microspheres). Liposomal encapsulation may be used and the liposomes may be derivatized with various polymers.

Appropriate dosage levels for treatment of various conditions in various patients will depend on the patient and the therapeutic purpose. Generally, for injection or infusion, dosage will be between 0.01 μg of anti-MCEF reagent/kg body weight and 10 mg/kg. (Similar numbers apply to other reagents against P-TEFb components, antiHSP90 or antiCDC37 reagent). The dosing schedule may vary, depending on the circulation half-life of the reagent (or its precursor) whether, for example, the inhibitor is delivered by bolus dose or continuous infusion, and the formulation used.

### Example 11

In order to test if our model of P-TEFb recruitment by MCEF is correct, we first performed western blots on various P-TEFb preps with anti-MCEF, and found that MCEF was present. We then performed the opposite immunoprecipitations. Antisera directed against the rMCEF 1-715 and rMCEF 1-300 proteins expressed in E. Coli, were coupled to protein-A sepharose by standard techniques and crosslinked with DMP. The crosslinked antisera and equivalently crosslinked pre-immune sera were used in immunoprecipitations from nuclear extracts. The immunoprecipitates were then western bloted against antisera to components of P-TEFb. Consistently, in 5 different experiments, CDK9, Cyclin T1 and MCEF were found to co-immunoprecipitate. A sample immunoprecipitation bloted with antisera directed against CDK9 is shown in FIG. 8. Clearly, MCEF co-immunoprecipitates with P-TEFb. Combined with our original detection of MCEF in P-TEFb by sequencing, we conclude that MCEF is a component of P-TEFb.

### Example 12

Hybridization

Many methods of hybridizing a probe nucleic acid molecule with a target molecule are known. Generally, a person will select conditions that are optimized for the length and base sequence of the two molecules being hybridized. When the hybridized complex is accessible by nucleases, appropriate enzymes are available for eliminating mismatched unhybridized regions. According to procedures well-established in the art, the procedure will be varied according to whether the process is intended to screen clones for specific mRNA sequences, detect nucleic acid molecules that are the result of PCR amplification, locate DNA sequences on a chromosome; whether the nucleic acid target is in a biological cell, and numerous other possibilities.

The book, Molecular Cloning, A Laboratory Manual, Second Edition, J. Sambrook et al., editors, Cold Spring Harbor Laboratory Press, (1989) describes hybridization conditions, including those suitable for screening cDNA libraries. The Manual points out that for DNA molecules more than 200 nucleotides in length, hybridization is usually carried out at 15-25° C. below the calculated melting temperature (Tm) of a perfect hybrid. For short oligonucleotides, on the other hand it is preferred to carry out hybridization at 5-10° C. below the Tm. The Manual also cites as useful an equation for calculating Tm,

Tm=81.5-16.6(log₁₀[Na+])+0.41(%G+C)−(600/N)

where N is the chain length in nucleotides, for oligonucleotides as long as 60-70 nucleotides or as short as 14 nucleotides.

Fluorescence in situ hybridization can be done using, for example, using biotinylated probes to detect mRNA. DNA chip hybridization can be used to measure mRNA levels. Detection of mRNA molecules, including those with translocation junctions, can also be achieved using PCR. Hybridization of RNA probes to purified DNA preparations is also possible.

### Example 13

Isolation of a cell line stably expressing epitope tagged CDK9

In order to address the question of what proteins may associate intracellularly with P-TEFb, we constructed a stable FLAG-tagged CDK9 (f:CDK9)-expressing HeLa cell line from which we could then prepare extracts and irnmunoprecipitate CDK9-associated proteins. As controls we used HeLa cells and cell lines stable transfected with f:DSIF p160, f:CDK9M (containing a mutant CDK9 kinase activity, see Materials & Methods) and the empty vector (pCIN4). Clones selected were shown to express epitope-tagged proteins of circa 45 kDa for the f:CDK9 or f:CDK9M cell lines, and of circa 160 kDa for the f:DSIF p160 cell line. The expected sizes for DSIF p160 (Prasad et al., 1995) and for CDK9 or CDK9M (Grana et al., 1994) are 160 and 43 kDa, respectively. The f:CDK9 and f:CDK9M cell lines selected for this study expressed similar levels of tagged proteins.

### Example 14

Immunopurified FLAG-tagged CDK9 preparations contain the known P-TEFb components

Immunopurified FLAG-tagged CDK9 preparations contain the components expected for p-TEFb. From nuclear extracts of the f:CDK9 cell line, we immunopurified f:CDK9 and associated proteins. The f:CDK9 preparation that was immunopurified at high salt (500 mM KCl) contained f:CDK9, but not the faster migrating and slightly smaller endogenous (un-tagged) CDK9. This indicates that f:CDK9 does not associate with the endogenous CDK9 and is consistent with previous work showing that CDK9 essentially acts as a monomer within the complex (O'Keeffe et al., 2000). The endogenous CDK9 was more abundant in control HeLa cell nuclear extracts than in nuclear extracts from the f:CDK9-expressing cells, consistent with a previous observation that cell lines expressing epitope-tagged CDK9 exhibit down regulation of endogenous CDK9 (O'Keeffe et al., 2000). The f: CDK9 preparation immunopurified at high salt (300 mM KCl) also specifically contained cyclin T1 in addition to f:CDK9, consistent with the known composition of natural P-TEFb (Wei et al., 1998; Yang et al., 1997).

An association of HSP90, HSP70 and CDC37 proteins with P-TEFb preparations was recently reported (O'Keeffe et al., 2000). There are two isoforms of HSP90 and, by comparison to recombinant a and b forms, it appears that HSP90a is the predominant form found both in nuclear extracts and in f:CDK9 preparations immunopurified at high salt (500 mM KCl) Antibodies directed against HSP90a recognize both isoforns and antibodies, while antibodies directed against HSP90b are specific for b. We also detected HSP70 and CDC37 as specificaly associated proteins in f:CDK9 preparations that were immunopurified at low salt (100 mM KCl). Although nucleolin has been reported to be in a novel holoenzyme complex containing cyclin T1, CDK9 and Pol II (Parada and Roeder, 1999), it was not detected, even at low salt (100 mM), in immunopurified preparations of f:CDK9. This suggests that the P-TEFb-associated proteins detected here, anchored directly or indirectly to f:CDK9, are distinct from such a holoenzyme form.

### Example 15

Immunopurified FLAG-tagged CDK9 preparations contain the kinase activity expected for P-TEFb

In order to test whether the immunopurified f:CDK9 preparation contained P-TEFb activity, a high salt (500 mM KCl) preparation was assayed for its ability to phosphorylate known substrates of P-TEFb in vitro. As substrates we used a recombinant glutathione-S-transferase (rGST)-fusion protein containing a portion of the CTD of the largest (220 kDa) subunit (RPB1) of pol II, RPB1 itself, and an immunopurified f:DSIF p160 preparation. The f:CDK9 immunopurified preparation was able to phosphorylate both rGST-CTD and RPB1 within Pol II. As reported by others for P-TEFb (Ivanov et al., 2000; Kim and Sharp, 2001), the immunopurified f:CDK9 actively phosphorylated a circa 160 kDa polypeptide, most likely f:DSIF p160, within an immunopurified f:DSIF p160 preparation. This f:DSIF p160 phosphorylation was not the result of an associated kinase (such as endogenous P-TEFb) in the f:DSIF p160 preparation, because only a very marginal amount of f:DSIF p160 was phosphorylated in a control kinase reaction containing only the f:DSIF p160 preparation. The control immunopurified f:CDK9M preparation (see Materials and Methods) was significantly impaired in its ability to phosphorylate either rGST-CTD or RPB1 within Pol II and was weaker in its ability to phosphorylate the 160 kDa polypeptide in the f:DSIF p160 preparation. These results implicate f:CDK9 as the kinase in the f:CDK9 preparation. Taken together with the previous immunological identification of P-TEFb components in the f:CDK9 immunopurified preparation, we conclude that we have likely immunopurified an enzymaticaly active human epitope-tagged P-TEFb and associated proteins (HSP90, CDC37, HSP70 and possibly others).

### Example 16

Immunopurified epitope-tagged P-TEFb contains a novel associated protein

The polypeptide compositions of f:CDK9 P-TEFb and control preparations (from cells expressing only the pCIN4 vector) immunopurified at various salt concentrations (270, 350, and 500 mM KCl) were analyzed by SDS-PAGE and silver staining. A group of polypeptides specific for the f:CDK9 preparations was increasingly apparent at higher salt concentrations; at 500 mM KCl, an identical pattern of polypeptides was also observed for the f:CDK9M immunopurified preparation and for a f:CDK9 preparation immunopurified from cytoplasmic extracts. Bands that appeared to be specifically associated at 500 mM KCl were excised from a preparative SDS-PAGE Coomassie blue stained gel and microsequenced. The sequence data identified CDK9, cyclin T1, cyclin T2A, cyclin T2B, HSP90, CDC37, and a novel protein containing the peptide sequence (VGPAPSTSQSQK) as specific components. EST database searches with the novel peptide sequence identified previously uncharacterized ESTs that matched this sequence. Because this novel protein purified with CDK9 at high salt concentration, it was named MCEF for Major CDK9 Elongation Factor-associated protein.

### Example 17

Cognate cDNA cloning and sequence analyses indicate that MCEF is expressed in many human tissues and is a new AF4/FMR2 family member

The cloning of MCEF through a combination of RACE and screening of a fetal brain Lambda cDNA library is described in Materials and Methods. The final clone encompassed 5,856 bp. (submitted to GenBank). This was used to generate a probe and to translate a putative protein from a major open reading frame. The cDNA contains an ATG at position 30, and is surrounded by a strong Kozak sequence (Kozak, 1999); however, this start position is followed by an amber in-frame termination codon at position 42. Based on re-sequencing of this position in several distinct clones, we believe that the start codon is actually at position 66, surrounded by a weak Kozak sequence. Database searches suggest that MCEF represents a new member of the AF4/FMR2 family of transcription factors. A long theoretical 3′-untranslated region (UTR) was found. The MCEF cDNA that we obtained contains 2,696 bp of the 3′-UTR, but lacks a poly A sequence. Comparison with other family member cDNA sequences shows that there is good overall cDNA sequence homology for coding sequences (approximately 50%) and slightly lower homology over non-coding sequences (40%). Long 3′-UTRs are not unusual in this family. A major 10 kb transcript was detected for MCEF mRNA in all tissues examined (bone marrow, peripheral blood lymphocytes, thymus, lymph nodes, spleen, pancreas, kidney, liver, lung, placenta, skeletal muscle, and fetal liver), although at least 3 minor transcripts of approximately 5, 7 and 8 kb also appear to be expressed. Further database analysis identified MCEF genomic sequences on chromosome 5, near the interleukin gene cluster and within Alu repeat sequences . Preliminary analysis indicates that MCEF mRNA is encoded by as many as 16 exons that span 50 kb.

Between the start codon and an opal stop codon lies a 3,489 bp open reading frame encoding a 1,163 amino acid protein with a molecular weight of 127 kDa. The theoretical MCEF protein is 18.83% S, 10.49% K and 8.08% P, which is not unusual for this family. The theoretical MCEF protein shows significant homology with AF4 around the mapped AF4 strong transactivation domain (Prasad et al., 1995), as well as significant homology in the N-tenninal region with all Af4 and FMR2 family members. However, the peptide sequence derived from the original immunopurified f:CDK9 P-TEFb preparation corresponds only to MCEF. Phylogenetic analysis indicates that hMCEF, hAF4, hFMR2, hLAF and mLAF proteins are an outgroup from the next nearest sequence in the Swiss-Protein database. This demonstrates that these sequences indeed form a distinct family. We conclude that the novel human f:CDK9 P-TEFb-associated protein is very likely the translation product of a widely expressed 10 kb MCEF mRNA that is encoded by an MCEF gene near the interleukin gene cluster on chromosome 5.

### Example 18

Antisera directed against recombinant MCEF specifically immunoprecipitate P-TEFb from nuclear extracts

In order to confirm that MCEF is naturally associated with P-TEFb and not just with free over-expressed f:CDK9, we first generated polyclonal antisera to recombinant MCEF (rMCEF) that was expressed in, and purified from, E. Coli. Antisera from rMCEF immunized rabbits specifically recognized several polypeptides that included a major 140 and a 55 kDa species in nuclear extracts. Antisera directed against amino acids 1-715 of rMCEF were able to immunoprecipitate both f:CDK9 and natural endogenous CDK9 from f:CDK9 nuclear extracts and to specifically immunoprecipitate natural CDK9 from control nuclear extracts. If MCEF were only associating with free CDK9, antisera directed against rMCEF should not immunoprecipitate cyclin T. However, antisera directed against either amino acids 1-715 or amino acids 1-420 of rMCEF immunoprecipitated cyclin T₁ as well as CDK9. An additional indication that MCEF co-immunoprecipitates with P-TEFb is that antisera directed against amino acids 1-715 of rMCEF co-immunoprecipitated an MCEF doublet as well as cyclin T₁. Finally, antibodies directed against amino acids 1-715 of rMCEF also detected MCEF in the immunopurified f:CDK9 P-TEFb preparation but not in a control immunoprecipitate.

### Example 19

MCEF represses HIV-1 replication and appears to repress transcription when directly tethered to a promoter

In order to determine if the MCEF cDNA can drive expression of the theoretical 127 kDa MCEF protein, we subcloned the MCEF cDNA open reading frame into a T7 promoter-containing vector (see Material and Methods). Interestingly, whereas no product was expressed with the full-length cDNA (data not shown), removal of the first 66 bp resulted in expression in reticulocyte lysates of a circa 140 kDa protein, in good agreement with the theoretical 127 kDa . Similarly, a cDNA (G4DBD-MCEF) expressing full length MCEF fused to the 141 amino acid Gal4 DNA binding domain was able to drive the expression of a protein with a slightly reduced migration relative to MCEF.

In order to examine the potential effect of MCEF on HIV-1 replication, we examined in vivo effects of ectopic expression in HeLa cells. Ectopic expression of MCEF represses HIV replication in HeLa cells. Using a single-round replication assay measuring HIV-1 p24 antigen levels at 24 hours post transfection (Naghavi et al., 1999), increasing amounts of MCEF led to a dose dependent repression that was as high as ten fold. However, an obvious toxic effect was observed at higher concentrations (3, 5 and 7 mg) of co-transfected DNA. In addition, because of the significant size difference between the effector (MCEF) construct and the empty vector, at equimolar plasmid concentrations empty vector transfections always contained less total DNA than effector transfections. Therefore, we made the identical size antisense construct (as a negative control) and then compared the effects of sense versus antisense MCEF-expressing vectors at a non-toxic concentration of 2 mg (now at equimolar concentrations). Remarkably, over the background of endogenous MCEF levels, a 60% repression of HIV-1 replication was still observed within 24 hours.

A previous report showed that a fusion protein between the Gal4 DNA binding domain and AF4 or FMR2 (two members of the family to which MCEF belongs) activated transcription from a promoter containing 5 Gal4 DNA binding sites (Prasad et al., 1995). We therefore determined if MCEF could also function as an activator in such a context. However, an analysis with the full length pcG4DBD-MCEF SENSE failed to show any activation by MCEF when tethered to such a promoter, consistent with a repressive role for MCEF.

In the previous experiment the basal level of transcription from the minimal promoter was too low to register a potential repressive effect of the G4-MCEF fusion. Therefore we repeated the experiment with a vector containing an enhancer upstream of the Gal4 binding sites (see Materials and Methods). The G4MCEF sense construct showed a small but significant repressive effect when compared to the control G4MCEF antisense construct, whereas the MCEF sense construct did not show any repression when compared to the control MCEF antisense construct.

Ectopic expression of MCEF represses transcription in HeLa cells when tethered to a promoter. Reporter constructs driving the expression of the luciferase cDNA open reading frame were co-transfected into HeLa cells. The co-transfected vectors drove the expression of the MCEF open reading frame (MCEF-SENSE), the MCEF open reading frame cloned in a reverse orientation (MCEF-ANTISENSE), fusions between the 141 amino acids of the Gal4 DNA binding domain and the MCEF open reading frame (G4MCEF-SENSE) or a similar fusion with the MCEF open reading frame cloned in a reverse orientation (G4MCEF-ANTISENSE). A minimal promoter containing a TATA box and 5 Gal4 target binding sequences cloned in front of the luciferase cDNA (pfrLuc) was used in transient transfections with the indicated co-transfected constructs. Co-transfection of a construct lacking an activation domain (pCMV-BD) was used as a negative control. Constructs able to drive the expression of fusion proteins between the 141 amino acid Gal 4 DNA binding domain and the activation domain of NFkB (G4NIFkB) or the activation domain of VP16 (G4VP16) were used as positive controls. The reporter used in A was modified to contain CMV enhancer sequences upstream of the 5 Gal 4 DNA binding sites (raising the level of luciferase expression). A transfection without a co-transfected construct was used as a negative control (NO EFFECTOR).

Discussion

DSIF contains p160 and p14. DSIFp160 is target for the nucleoside analogue DRB, in conferring DRB sensitivity to elongation [26, 33, 34]. It was later found that in addition to DSIF, a multiple polypeptide component NELF was required for DRB sensitivity [17]. Interactions with several proteins including Pol ii and DSIFp14 (hSPT4) have been reported for DSIFp160 [18]. In our hands, the FT-DSIFp160 immunopurified material was found to contain no additional specific components other than DSIF-p14. It is entirely possible that at less stringent conditions, sub-equimolar amounts of the reported DSIF-p160-interacting proteins could be present at our lower salt concentrations (100 mM), but we have not investigated this possibility, since our interest was in determining strongly interacting components of complexes for DSIF and P-TEFb.

P-TEFb regulation

The chaperone pair Cdc37-HSP90 is frequently found with kinases [19]. The finding of Cdc37 and HSP90 in our P-TEFb preparation may indicate a functional nuclear role for these components, however a more likely possibility is that we have identified the involvement of the Cdc37-HSP90 chaperone pair pathway in the regulated assembly of P-TEFb. Either way, our finding suggests that the Cdc37-HSP90 proteins may represent novel targets for anti-P-TEFb, or anti-HIV therapies. Although our preliminary results indicated a profound repressive effect of Geldanamycin (GA), a specific HSP90 inhibitor, upon both Tat-transactivation and HIV-1 viral replication, unfortunately these effects appear to be primarily the result of GA cytotoxicity. We were unable to isolate P-TEFb from stable Cyclin T expressing cell lines. Indeed, transfection of FT-CyclinT constructs into HeLa-S3 cell lines was highly toxic and the few neomycin resistance clones did not express Ft-Cyclin T.

P-TEFb contains the novel gene product MCEF

The components of P-TEFb have been shown in several papers as comprising several previously uncharacterized bands on SDS-PAGE analysis; (Reviewed in: [7] and in [3]) [13] [9, 12]. Here we have determined that one of these bands contains a protein from which a peptide sequence was used to clone the novel gene MCEF. It could be argued that the original protein band from which the peptide sequence was derived represents a contaminant in our P-TEFb preparation. In support of this line of argument is the apparent discrepancy between the circa 50 kD molecular weight of the band from which the peptide was sequenced and the 85 kD major ORF within the MCEF sequence, aa.1-715 (or the 140 kD major ORF within the MCEF 1-1163 aa). However, that MCEF is not a specific component of P-TEFb seems unlikely because all of the other derived peptide sequences for the 500 mM FT-CDK9 complex were found to be either confirmed components of P-TEFb (Cyclin T1, T2A, T2B, CDK9) or logical components for a kinase-containing complex (HSP90-Cdc37). More likely explanations for the discrepancy between the size of MCEF predicted from the major ORF and the size of the protein band from our P-TEFb preparation, are either post-translational processing or differential splicing, both having ample antecedents in the literature. In this regard, it is noteworthy that in several tissues, particularly heart, there are smaller transcripts detected with an MCEF probe (FIG. 4B). In addition, it is also noteworthy that in P-TEFb preparations detecting a 140 l band, a 50 kD band is absent and vice versa, suggesting these two may be related forms of MCEF. Indeed, FIG. 6 shows clearly that in Hela S cells, from where the MCEF peptide sequence was derived, there was a p140 and a p55 protein band.

MCEF represents a novel member of the AF4/FMR2 family of transcription factors

AF4 is likely involved in disregulation of transcription in leukemias [21, 35, 36]. One likely possibility is a gain of function, because the chimeric MLL-AF4 protein contains a strong transactivation domain of AF4 [29]. FMR2 is involved in mild mental retardation as a result of amplified repeats, methylation and gene silencing of the FMR2 gene, thus a loss of function [37]. It is interesting that two apparently disparate diseases have genes implicated from the same family of transcription factors, indicating a possible mechanistic convergence (see below). That MCEF is a novel member of the AF4/FMR family of transcription factors is supported strongly by the phylogenetic analysis and that there may be a common mechanism behind the actions of this family of transcription factors is further suggested by the conservation of the transactivation domain.

MCEF is a candidate gene for involvement in leukemias involving chromosome 5 abnormalities. Chromosome 5 abnormalities have been repoited to be associated with ALL [22]. We propose here that because of the chromosomal location of MCEF, it represents a candidate gene breakpoint for these chromosome abnormalities. It will be interesting to determine if MCEF can be found fused to MLL, as for AF4 in leukemias [21, 35, 36]. If this is the case, then because of their similarities in transactivation domains, AF4, MCEF and FMR2 may share a similar mechanism of action at promoters, via recruitment of similar factors, such as P-TEFb.

A model for a common mechanism for disregulation of gene expression in HIV infection and leukemias involving AF4/FMR2 family members

AF4 was first described as a fusion partner for the MLL protein in ALL, stemming from the most frequent chromosome abnormalityin childhood leukemias, a (4:11) translocation leukemias [21, 35, 36]. Although there is intense interest in ALL, the mechanism by which this translocation may be the cause of ALL, remains to be determined [38]. What is known is that AF4-GAL4 fusions ectopically expressed can transactivate transcription ten fold from a GAL4-binding-site-reporter, in transient transfections [29]. This same analysis was used to roughly map a strong trans-activation domain of AF4 to aa 480-560. Importantly, this region is highly conserved in MCEF, as well as in FMR2 family member and in MLL-AF4 fusions. MLL has been shown to interact with SWI/SNF family members, via its C-terminal end. Because this region is lost in the MLL-AF4 translocation fusion protein [39], one possibility for the mechanism of action of MLL-AF4 would be loss of function in MLL/ALL-1. However, our finding that a component of P-TEFb is a family member of AF4, suggests that if MLL-MCEF fusions are found, then the mechanism of action of these fusion proteins could be abnormal recruitment of P-TEFb to MLL targets, a gain of function (FIG. 7). The similarity with Tat recruitment is evident from FIG. 7. In support of this model, it should be noted that precedent for abnormal recruitment of an elongation factor, via translocation and production of a fusion protein has been reported, in the form of MLL-ELL [40]. In this respect, leukemias resulting from MLL-AF4 or chromosome 5 abnonnalities (for which MLL-MCEF are to be expected), and AIDS, would both be considered diseases of abnormal elongation factor recruitment.

We have made an epitope-tagged CDK9 expressing cell line from which we immunopurified P-TEFb and identified a novel P-TEFb-associated protein named MCEF. We further cloned the corresponding cDNA and showed that MCEF can function as a repressor of HIV-1 gene expression.

By three different techniques we have shown that our immunopurified epitope-tagged preparation is P-TEFb. First, western blots revealed that previously identified P-TEFb or P-TEFb-associated proteins (O'Keeffe et al., 2000) were specifically present in our preparation. Secondly, we used an in vitro kinase assay as an orthogonal confirmation that we have purified an activity consistent with that of P-TEFb. This revealed, as well, that our preparation can actively phosphorylate DSIF p160 within the context of a similarly immunopurified DSIF. Since several groups have now shown that DSIF p160 is a specific substrate for CDK9 (Ivanov et al., 2000; Kim and Sharp, 2001), this further indicated that our immunopurified preparation contains P-TEFb. Finally, we directly sequenced proteins that appeared to interact specifically at high stringency (500 mM KCl) in our immunopurified preparation, confirming directly that the expected components of P-TEFb were indeed contained in our preparations.

The chaperone pair CDC37-HSP90 is frequently utilised for kinase folding (Buchner, 1999). Our finding of HSP90-CDC37 in association with P-TEFb confirms a similar report (O'Keeffe et al., 2000) which further suggested that inhibitors of HSP90 may block HIV replication. However, ourpreliminary attempts to target this chaperone pair with geldanamycin (an inhibitor of HSP90) only showed an inhibition of HIV-1 replication or LTR-directed transcription at cytotoxic concentrations.

Previous published preparations of human P-TEFb contained a number of proteins other than p87 (cyclin T1) (Wei et al., 1998) and p43 (CDK9) (Yang et al., 1997). For example, specific bands of circa 40, 87, 105, 133, 140, and 207 kDa were detected by SDS-PAGE in some preparations (Zhu et al., 1997), whereas bands of circa 43, 49, 55, 61, 68, and 87 kDa were apparent in other preparations (Zhou et al., 1998). The polypeptide composition differences may be the result of different methodologies that did not employ the CDK9 epitope-tagging and immuno-affinity purification procedures used here. In our affinity purified P-TEFb preparations isolated under stringent salt (500 mM KCl) conditions, we consistently found specific bands of p43, p55, p85, p87 and p90 and have identified them by direct sequencing. It should be noted, however, that although other proteins may specifically associate with P-TEFb at lower (more physiological) salt concentrations, we have focused here only on tightly-associated proteins. Under these stringent conditions, we have determined that one of the P-TEFb-associated proteins is MCEF.

It was at first surprising to find that the specifically detected circa 55 kDa polypeptide corresponded to a protein that could be classified as a new member of the AF4 family. However, the finding that MCEF is expressed in a wide range of tissues is consistent with a general role for a protein associated with an elongation factor such as P-TEFb. In addition, while this work was in preparation, Taki et. al. (1999) have reported a novel gene., AF5, that is fused to MLL in a single leukemic patient and nearly identical in sequence to MCEF (Taki et al., 1999). Furthermore, precedent for abnormal recruitment of an elongation factor via translocation and production of a fusion protein has been set by the description of the MLL-ELL fusion (Shilatifard et al., 1996). Therefore it is perhaps not surprising that another elongation factor (P-TEFb) is also involved with the novel AF4 family member MCEF.

After cloning the MCEF cDNA, we were further surprised that the full-length cDNA contained a much larger open reading frame than the circa 55 kDa polypeptide from which we obtained the MCEF peptide sequence. Because all the other peptide sequences obtained from specific bands in our immunopurified P-TEFb preparation have been shown to be associated with P-TEFb by other groups, it is highly likely that the observed association of MCEF with P-TEFb is physiological and of functional significance rather than adventitious. One possibility is that the p55 kDa band, which provided the MCEF peptide sequence, is either an alternatively spliced version of MCEF or a processed form of p140 MCEF. Precedent for alternative splicing occurs in the AF4/FMR2 family since OX19 is a 50 kDa alternative splice product of the 124 kDa FMR2 (Gecz et al., 1997). Another possibility not yet ruled out is that during the immunopurification process a native 140 kDa P-TEFb-associated MCEF is rapidly degraded to the 55 kDa protein. Interestingly, some P-TEFb preparations of other groups have been shown to have either an associated 140 kDa polypeptide without a 50 kDa polypeptide (Zhu et al., 1997) or an associated 50 kDa polypeptide without a 140 kDa polypeptide (Zhou et al., 1998). Whatever the exact relationship between the MCEF p140 and p55 proteins, the fact that we were able to specifically immunoprecipitate both cyclin T1 and CDK9 with antisera directed against MCEF, and that we were able to detect MCEF in immunopurified P-TEFb preparations, is strong evidence that some form of MCEF is naturally associated with P-TEFb.

As for other regions of the HIV-1 genolie (Estable et al., 1998b), LTRs of different subtypes or variants within a subtype can differ in their sequence and transcription abilities (Estable et al., 1998a; Estable et al., 1996; Estable et al., 1999; Naghavi et al., 2001; Naghavi et al., 1999). In HeLa cells, MCEF has a modest repressive effect on Tat-transactivation in the context of a transiently transfected HIV-1 subtype B LTR. In contrast, we detected a significantly strong inhibition of HIV-1 in single round replication assays also using a subtype B LTR. It will be interesting to see if this repressive effect is observed with other HIV-1 subtypes.

At this time we do not know the level at which MCEF acts to repress HIV-1 in the context of the replication assay. However, given that MCEF is associated with P-TEFb at high KCl concentrations and that it represses rather than activates when tethered to an otherwise elongation competent promoter, we suspect that it is acting at a superimposed level of regulation over P-TEFb. Further experiments will be required to more clearly dissect the mechanism of action.

References

1. Roeder, R. G. and W. J. Rutter, Multiple forms of DNA-dependent RNA polymerase in eukaryotic organisms. Nature, 1969. 224(216): p. 234-7.

2. Hampsey, M., Molecular genetics of the RNA polymerase II general transcriptional machinery. Microbiol Mol Biol Rev, 1998. 62(2): p. 465-503.

3. Karn, J., Tackling Tat. J Mol Biol, 1999. 293(2): p. 235-54.

4. Wei, P., et al., A novel CDK9-associated C-type cyclin interacts directly with HV-1 Tat and mediates its high-affinity, loop-specific binding to TAR RNA. Cell, 1998. 92(4): p. 451-62.

5. Chen, D. and Q. Zhou, Tat activates human immunodeficiency virus type 1 transcriptional elongation independent of TFIIH kinase. Mol Cell Biol, 1999. 19(4): p. 2863-71.

6. Isel, C. and J. Karn, Direct evidence that HIV-1 Tat stimulates RNA polymerase II carboxyl-terminal domain hyperphosphorylation during transcriptional elongation. J Mol Biol, 1999. 290(5): p. 929-41.

7. Jones, K. A., Taking a new TAK on tat transactivation [comment]. Genes Dev, 1997. 11(20): p. 2593-9.

8. Marshall, N. F. and D. H. Price, Control of formation of two distinct classes of RNA polymerase II elongation complexes. Mol Cell Biol, 1992. 12(5): p. 2078-90.

9. Marshall, N. F. and D. H. Price, Purification of P-TEFb, a transcription factor required for the transition into productive elongation. J Biol Chem, 1995. 270(21): p. 12335-8.

10. Marshall, N. F., et al., Control of RNA polymerase II elongation potential by a novel carboxyl-terminal domain kinase. J Biol Chem, 1996. 271(43): p. 27176-83.

11. Herrmann, C. H. and A. P. Rice, Lentivirus Tat proteins specifically associate with a cellular protein kinase, TAK, that hyperphosphorylates the carboxyl-terminal domain of the large subunit of RNA polymerase II: candidate for a Tat cofactor. J Virol, 1995. 69(3): p. 1612-20.

12. Zhu, Y., et al., Transcription elongation factor P-TEFb is required for HIV-1 tat transactivation in vitro. Genes Dev, 1997. 11(20): p. 2622-32.

13. Zhou, Q., et al., Transcription elongation factor P-TEFb mediates Tat activation of HIV-1 transcription at multiple stages. Embo J, 1998. 17(13): p. 3681-91.

14. Yang, X., et al., TAK, an HIV Tat-associated kinase, is a member of the cyclin-dependent family of protein kinases and is induced by activation of peripheral blood lymphocytes and differentiation of promonocytic cell lines. Proc Natl Acad Sci U S A, 1997. 94(23): p. 12331-6.

15. Peng, J., N. F. Marshall, and D. H. Price, Identification of acyclin subunit required for the function of Drosophila P-TEFb. J Biol Chem, 1998. 273(22): p. 13855-60.

16. Peng, J., et al., Identification of multiple cyclin subunits of human P-TEFb. Genes Dev, 1998. 12(5): p. 755-62.

17. Yamaguchi, Y., et al., NELF, a multisubunit complex containing RD, cooperates with DSIF to repress RNA polymerase II elongation. Cell, 1999. 97(1): p. 41-51.

18. Yamaguchi, Y., et al., Structure and function ofthe human transcription elongation factor DSIF. J Biol Chem, 1999. 274(12): p. 8085-92.

19. Stepanova, L., X. Leng, and J. W. Harper, Analysis of mammalian Cdc37, a protein kinase targeting subunit of heat shock protein 90. Methods Enzymol, 1997. 283: p. 220-9.

20. Gecz, J., et al., Gene structure and subcellular localization of FMR2, a member of a new family of putative transcription activators. Genomics, 1997. 44(2): p. 201-13.

21. Li, Q., J. L. Frestedt, and J. H. Kersey, AF4 encodes a ubiquitous protein that in both native and MLL-AF4 fusion types localizes to subnuclear compartments. Blood, 1998. 92(10): p. 3841-7.

22. Heerema, N. A., et al., Cytogenetic studies of infant acute lymphoblastic leukemia: poor prognosis of infants with t(4;11)—a report of the Children's Cancer Group. Leukemia, 1999. 13(5): p. 679-86.

23. O'Keeffe, B., et al., Requirement for a kinase-specific chaperone pathway in the production of a Cdk9/Cyclin T1 heterodimer responsible for P-TEFb-mediated tat stimulation of HIV-1 transcription [In Process Citation]. J Biol Chem, 2000. 275(1): p. 279-87.

24. Taki, T., et al., AF5q31, a newly identified AF4-related gene, is fused to MLL in infant acute lymphoblastic leukemia with ins(5;11)(q31;q13q23). Proc Natl Acad Sci U S A, 1999. 96(25): p. 14535-40.

25. Grana, X., et al., PITALRE, a nuclear CDC2-related protein kinase that phosphorylates the retinoblastoma protein in vitro. Proc Natl Acad Sci U S A, 1994. 91(9): p. 3834-8.

26. Wada, T., et al., DSIF, a novel transcription elongation factor that regulates RNA polymerase II processivity, is composed of human Spt4 and Spt5 homologs. Genes Dev, 1998. 12(3): p. 343-56.

27. Rees, S., et al., Bicistronic vector for the creation of stable mammalian cell lines that predisposes all antibiotic-resistant cells to express recombinant protein. Biotechniques, 1996. 20(1): p. 102-4, 106, 108-10.

28. Dignam, J. D., R. M. Lebovitz, and R. G. Roeder, Accurate transcription initiation by RNA polymerase II in a soluble extract from isolated mammalian nuclei. Nucleic Acids Res, 1983. 11(5): p. 1475-89.

29. Prasad, R., et al., Domains with transcriptional regulatory activity within the ALL1 and AF4 proteins involved in acute leukemia. Proc Natl Acad Sci U S A, 1995. 92(26): p. 12160-4.

30. Estable, M. C., et al., Naturally occurring human immunodeficiency virus type 1 long terminal repeats have a frequently observed duplication that binds RBF-2 and represses transcription. J Virol, 1998. 72(8): p. 6465-74.

31. Estable, M. C., et al., Human immunodeficiency virus type long terminal repeat variants from 42 patients representing all stages of infection display a wide range of sequence polymorphism and transcription activity. J Virol, 1996. 70(6): p. 4053-62.

32. Estable, M. C., et al., Purification of RBF-2, a transcription factor with specificity for the most conserved cis-element of naturally occurring HIV-1 LTRs. J Biomed Sci, 1999. 6(5): p. 320-32.

33. Yamaguchi, Y., T. Wada, and H. Handa, Interplay between positive and negative elongation factors: drawing a new view of DRB. Genes Cells, 1998. 3(1): p. 9-15.

34. Wada, T., et al., Evidence that P-TEFb alleviates the negative effect of DSIF on RNA polymerase II-dependent transcription in vitro. Embo J, 1998. 17(24): p. 7395-403.

35. Rubnitz, J. E. and C. H. Pui, Molecular diagnostics in the treatment of leukemia. Curr Opin Hematol, 1999. 6(4): p. 229-35.

36. Nasr, F., et al., Translocation t(4; 11)(q21;q23) and MLL gene rearrangement in acute lymphoblastic leukemia secondary to anti topoisomerase II anti-cancer agents. Leuk Lymphoma, 1997. 25(3-4): p.399-401.

37. Gu, Y., et al., Identification of FMR2, a novel gene associated with the FRAXE CCG repeat and CpG island. Nat Genet, 1996. 13(1): p. 109-13.

38. Cimino, G., et al., A prospective study of residual-disease monitoring ofthe ALL1/AF4 transcript in patients with t(4;11) acute lymphoblastic leukemia [In Process Citation]. Blood, 2000. 95(1): p. 96-101.

39. Rozenblatt-Rosen, O., et al., The C-tenninal SET domains of ALL-1 and TRITHORAX interact with the IN11 and SNR1 proteins, components of the SWI/SNF complex. Proc Natl Acad Sci U S A, 1998. 95(8): p. 4152-7.

40. Shilatifard, A., et al., An RNA polymerase II elongation factor encoded by the human ELL gene. Science, 1996. 271(5257): p. 1873-6.

What is claimed is:

1. A preparation comprising an MCEF-type polypeptide or an MCEF homolog, the preparation being substantially free of other human proteins, wherein an MCEF-type polypeptide is one that comprises at least 50 consecutive amino acids of the amino acid sequence of mammalian MCEF, said amino acid sequence being the one encoded by the MCEF cDNA insert contained in the plasmid deposited by ATCC accession number PTA-1502, and wherein an MCEF homolog is a sequence of length equal to an MCEF-type polypeptide, and having at least N percent identity with that polypeptide, N being 70.

2. A preparation of claim 1 wherein N is 80.

3. A preparation of claim 2 wherein N is 90.

4. A preparation of claim 1 that is an MCEF-type polypeptide.

5. A preparation of claim 4 wherein the MCEF-type polypeptide is one with the complete amino acid sequence encoded by the cDNA insert contained in the plasmid deposited under ATCC accession number PTA-1502.

6. A preparation of claim 1 wherein the MCEF-type polypeptide or MCEF homolog has been synthesized by recombinant DNA techniques.

7. A polypeptide comprising an immunogenic fragment of the MCEF-type protein encoded by the cDNA insert contained in the plasmid deposited under ATCC accession number PTA-1502, said fragment at least 7 amino acids in length.

8. A polypeptide of claim 7 wherein the fragment is at least 50 amino acids in length

9. A process of purifying an MCEF-type protein, said process comprising the steps of:
(a) isolating a P-TEFb complex; and
(b) purifying the MCEF-type protein from the P-TEFb complex.

10. A preparation of an MCEF-type protein purified by a process of claim 9.

11. An isolated MCEF nucleic acid molecule comprising (a) an MCEF base sequence that is at least 150 consecutive bases of, but preferably all of, the MCEF-coding base sequence of the cDNA insert in the plasmid deposited under ATCC accession number PTA-1502, or (b) a base sequence that is code-wise degenerate with such a MCEF base sequence, or (c) a base sequence that is a homolog of at least N per cent identity of such an MCEF base sequence, N being 70.

12. An isolated MCEF nucleic acid molecule of claim 11 wherein N is 90.

13. An isolated MCEF nucleic acid molecule of claim 11 comprising (a) an MCEF base sequence that is at least 150 consecutive bases of the MCEF-coding, base sequence of the cDNA insert in the plasmid deposited under ATCC accession number PTA-1502, or (b) a base sequence that is code-wise degenerate with such a MCEF base sequence.

14. An isolated MCEF nucleic acid molecule of claim 13 comprising (a) an MCEF base sequence that is the MCEF-coding base sequence of the cDNA insert in the plasmid deposited under ATCC accession number PTA-1502, or (b) a base sequence that is code-wise degenerate with such a MCEF base sequence.

15. An isolated MCEF nucleic acid molecule of claim 14 comprising an MCEF base sequence that is the MCEF-coding base sequence of the cDNA insert in the plasmid deposited under ATCC accession number PTA-1502.

16. A hybridization detection process for detecting MCEF nucleic acid, said process comprising the steps of:
a) hybridizing an MCEF hybridization probe against target nucleic acid so as to form a hybrid of said probe and said target; and
b) detecting said hybrid, wherein the MCEF hybridization probe comprises an MCEF nucleotide sequence or a nucleotide sequence complementary to an MCEF nucleotide sequence, or a homolog of at least N percent identity to either of said sequences, said MCEF nucleotide sequence comprising at least M contiguous nucleotides of the nucleotide sequence of the cDNA insert in the plasmid deposited under ATCC accession number PTA-1502, wherein M is 10 and wherein N is 70.

17. A process of claim 16 wherein N is 90.

18. A hybridization detection process of claim 16 wherein the MCEF hybridization probe comprises an MCEF nucleotide sequence or a nucleotide sequence complementary to an MCEF nucleotide sequence, said MCEF nucleotide sequence comprising at least M contiguous nucleotides of the nucleotide sequence of the cDNA insert in the plasmid deposited under ATCC accession number PTA-1502, wherein M is 10.

19. A hybridization translocation detection process for a leukemia chromosomal translocation, said process comprising the steps of:
a) hybridizing an MCEF hybridization probe against target nucleic acid spanning a translocation junction from a person or against nucleic acid generated by amplification procedures dependent on such a target nucleic acid as a template; and
b) detecting said hybrid, wherein the MCEF hybridization probe comprises an MCEF nucleotide sequence or a nucleotide sequence complementary to an MCEF nucleotide sequence, or a homolog of at least N percent identity to either of said sequences, said MCEF nucleotide sequence comprising at least M contiguous nucleotides of the nucleotide sequence of the cDNA insert in the plasmid deposited under ATCC accession number PTA-1502, wherein M is 10 and wherein N is 70.

20. A process of claim 19 wherein N is 90.

21. A hybridization translocation detection process of claim 19 wherein the MCEF hybridization probe comprises an MCEF nucleotide sequence or a nucleotide sequence complementary to an MCEF nucleotide sequence, said MCEF nucleotide sequence comprising at least M contiguous nucleotides of the nucleotide sequence of the cDNA insert in the plasmid deposited under ATCC accession number PTA-1502, wherein M is 10.

22. A DNA probe molecule or an RNA equivalent thereof, said DNA probe molecule between 14 and 70 nucleotides in length, which DNA probe molecule forms a hybrid with a DNA target molecule consisting of the base sequence of the cDNA insert in the plasmid deposited under ATCC accession number PTA-1502, or with a complement thereof, at a temperature 10° C. below Tm calculated according to the following equation
Tm=81.5−16.6(log₁₀[Na+])+0.41(%G+C)−(600/N)
where N is the chain length in nucleotides and where the hybridization is done in aqueous solution with the Na+concentration at 1 M, and wherein the G+C content refers to the probe.

23. A reagent binding assay for identifying a reagent that binds to an MCEF-type polypeptide, said process comprising the steps of:
a) reacting the reagent with the MCEF-type polypeptide; and
b) observing an affinity of the reagent for the MCEF-type polypeptide;
wherein an MCEF-type polypeptide is one that comprises at least 50 consecutive amino acids of the amino acid sequence of mammalian MCEF, said amino acid sequence being the one encoded by the MCEF cDNA insert contained in the plasmid deposited by ATCC accession number PTA-1502.

24. A process that is a leukemia-directed reagent-binding assay, the process comprising the steps of
(a) performing a reagent binding assay for a reagent that binds to a P-TEFb protein; and
(b) assaying for the effect of the reagent on the growth rate of leukemia cells, wherein said P-TEFb protein is selected from the group consisting of an MCEF-type polypeptide, CDK9, CDC37, HSP90, Cyclin T1, Cyclin T2A and Cyclin T2B, wherein an MCEF-type polypeptide is one that comprises at least 50 consecutive amino acids of the amino acid sequence of mammalian MCEF, said amino acid sequence being the one encoded by the MCEF cDNA insert contained in the plasmid deposited by ATCC accession number PTA-1502.

25. A process of claim 24, said process further comprising a step (c) of administering the reagent to a person suffering from leukemia.

26. A dissociation assay for reagents that will disrupt a P-TEFb complex to generate a P-TEFb protein unassociated with other P-TEFb complex members, said assay comprising treating a P-TEFb complex with said reagent and determining whether the P-TEFb protein has been disassociated, wherein said P-TEFb protein selected from the group consisting of an MCEF-type polypeptide, HSP90 and CDC37, wherein an MCEF-type polypeptide is one that comprises at least 50 consecutive amino acids of the amino acid sequence of mammalian MCEF, said amino acid sequence being the one encoded by the MCEF cDNA insert contained in the plasmid deposited by ATCC accession number PTA-1502.

27. An association inhibition assay for reagents that interfere with the ability of an P-TEFb protein to associate with one or more proteins, other than itself, of the P-TEFb complex, said P-TEFb protein selected from the group consisting of an MCEF-like polypeptide, HSP90, and CDC37, wherein an MCEF-type polypeptide is one that comprises at least 50 consecutive amino acids of the amino acid sequence of mammalian MCEF, said amino acid sequence being the one encoded by the MCEF cDNA insert contained in the plasmid deposited by ATCC accession number PTA-1502.

28. A process of administering a drug to a patient suffering from leukemia, said drug selected from the group consisting of (a) drugs that bind a P-TEFb protein, (b) drugs that dissociate P-TEFb to generate an unassociated P-TEFb protein; and (c) drugs that interfere with the ability of a P-TEFb protein to associate with one or more P-TEFb proteins, other than itself, wherein a P-TEFb protein is selected from the group consisting of an MCEF-type polypeptide, CDK9, Cyclin T1, Cyclin T2A, Cyclin T2B, HSP90, and CDC37. wherein an MCEF-type polypeptide is one that comprises at least 50 consecutive amino acids of the amino acid sequence of mammalian MCEF, said amino acid sequence being the one encoded by the MCEF cDNA insert contained in the plasmid deposited by ATCC accession number PTA-1502.

29. An MCEF-reactive antibody or serum, said antibody or serum being immunoreactive with an MCEF-like polypeptide, wherein an MCEF-type polypeptide is one that comprises at least 50 consecutive amino acids of the amino acid sequence of mammalian MCEF, said amino acid sequence being the one encoded by the MCEF cDNA insert contained in the plasmid deposited by ATCC accession number PTA-1502.

30. A diagnostic/prognostic assay wherein a sample of fluid, cells or tissue, said sample preferably blood or plasma, from a patient with leukemia, is reacted with an antibody or serum reactive with a P-TEFb protein to form an antibody-protein complex, said protein being a P-TEFb protein or a fusion protein comprising at least 10 consecutive amino acids of an MCEF-type polypeptide, such that the greater the amount of said complex, the poorer the prognosis for the patient in regard to leukemia, wherein the P-TEFb protein is selected firm the group consisting of an MCEF-type polypeptide, CDK9, Cyclin T1, Cyclin T2A, Cyclin T2B, HSP90 and CDC37, wherein an MCEF-type polypeptide is one that comprises at least 50 consecutive amino acids of the amino acid sequence of mammalian MCEF, said amino acid sequence being the one encoded by the MCEF cDNA insert contained in the plasmid deposited by ATCC accession number PTA-1502.

31. A process of claim 30 wherein the protein is a fusion protein.

32. A process that is a reagent-binding assay for identifying a reagent that binds to a P-TEFb protein and that is capable of interfering with HIV transcription or HIV replication, said process comprising the steps of:
a) reacting the reagent with the P-TEFb protein;
b) observing an affinity of the reagent for the P-TEFb protein;
c) adding the reagent to an assay for HIV transcription or HIV replication or said complex;
d) observing a decrease in the amount or transcript length in the assay for HIV transcription or observing a decrease in the amount of HIV replication in the assay for HIV replication;
wherein the P-TEFb protein is selected from the group consisting of an MCEF-type polypeptide, HSP90, and CDC37, and wherein the MCEF-type polypeptide is one that comprises at least 50 consecutive amino acids of the amino acid sequence of mammalian MCEF, said amino acid sequence being the one encoded by the MCEF cDNA insert contained in the plasmid deposited under ATCC accession number PTA-1502.

33. A process of claim 32 wherein the MCEF-type polypeptide is one with the complete amino acid sequence encoded by the cDNA insert contained in the plasmid deposited under ATCC accession number PTA-1502.

34. A process of claim 32, the process further comprising a step (5) of administering the reagent to a human subject suffering from HIV infection or AIDS.

35. A therapeutic process comprising the steps of:
(a) identifying a reagent as one that dissociates P-TEFb so as to generate an unassociated P-TEFb protein, said P-TEFb protein being selected from the group consisting of an MCEF-like polypeptide, HSP90, and CDC37.
(b) administering the reagent to a person who has AIDS or is HIV-infected, wherein the MCEF-type polypeptide is one that comprises at least 50 consecutive amino acids of the amino acid sequence of mammalian MCEF, said amino acid sequence being the one encoded by the MCEF cDNA insert contained in the plasmid deposited under ATCC accession number PTA-1502.

36. A therapeutic process comprising the steps of:
(a) identifying a reagent as one that interferes with the association of a first protein, with a second protein, said first protein selected from the group consisting of an MCEF-like polypeptide, HSP90, and CDC 37, said second protein selected from the group consisting of CDK9, cyclin T1, cyclin T2A, cyclin T2B, HSP90, and CDC37, and an MCEF-type polypeptide, provided said second protein is not the same as the first protein; and
(b) administering the reagent to a person who has AIDS or is HIV-infected, wherein the MCEF-type polypeptide is one that comprises at least 50 consecutive amino acids of the amino acid sequence of mammalian MCEF, said amino acid sequence being the one encoded by the MCEF cDNA insert contained in the plasmid deposited under ATCC accession number PTA-1502.

37. A process of administering a drug to a patient who is HIV-infected or suffers from AIDS, said drug selected from the group consisting of (a) drugs that bind to a P-TEFb protein, (b) drugs that dissociate P-TEFb to generate unassociated P-TEFb protein, and (c) drugs that interfere with the ability of the P-TEFb protein to associate with one or more proteins, other than itself, of the group consisting of CDK9, Cyclin T1, Cyclin T2A, Cyclin T2B, HSP90, CDC37, and an MCEF-type polypeptide, and wherein the P-TEFb protein is selected from the group consisting of an MCEF-type polypeptide, HSP90, and CDC 37, wherein the MCEF-type polypeptide is one that comprises at least 50 consecutive amino acids of the amino acid sequence of mammalian MCEF, said amino acid sequence being the one encoded by the MCEF cDNA insert contained in the plasmid deposited under ATCC accession number PTA-1502.

38. A process that is an assay system for testing the effects of a reagent on a P-TEFb protein's role in HIV transcription, said system comprising 3 components:
(a) DNA comprising the TAR region of HIV DNA and further comprising a transcribable region;
(b) A DNA-dependent RNA poymerase; and (c) purified P-TEFb protein or recombinant P-TEFb protein, such that components (a), and (b) and (c) are optionally in HIV-infected cells, wherein the P-TEFb protein is selected from the group consisting of an MCEF-type polypeptide, HSP90, and CDC 37, wherein the MCEF-type polypeptide is one that comprises at least 50 consecutive amino acids of the amino acid sequence of mammalian MCEF, said amino acid sequence being the one encoded by the MCEF cDNA insert contained in the plasmid deposited under ATCC accession number PTA-1502.

39. An MCEF nucleic acid hybridization assay for nucleic acid samples from individuals that are HIV-infected or AIDS-afflicted, said process comprising the steps of:
a) hybridizing an MCEF hybridization probe preparation against target nucleic acid from a person or against nucleic acid generated by amplification procedures from a person, said person being HIV-infected or AIDS-afflicted; and
b) quantifying the amount of hybridized MCEF;
wherein the MCEF hybridization probe comprises an MCEF nucleotide sequence or a nucleotide sequence complementary to an MCEF nucleotide sequence, or a homolog of at least N percent identity to either of said sequences, said MCEF nucleotide sequence comprising at least M contiguous nucleotides of the nucleotide sequence of the cDNA insert in the plasmid deposited under ATCC accession number PTA-1502, where M is 10 and wherein N is 70.

40. A hybridization assay of claim 39 wherein N is 90.

41. A hybridization assay of claim 39 wherein the MCEF hybridization probe comprises an MCEF nucleotide sequence or a nucleotide sequence complementary to an MCEF nucleotide sequence, said MCEF nucleotide sequence comprising at least M contiguous nucleotides of the nucleotide sequence of the cDNA insert in the plasmid deposited under ATCC accession number PTA-1502, where M is 10.

42. A hybridization assay of claim 41 wherein M is 20.

43. An MCEF nucleic acid hybridization assay for nucleic acid samples from individuals that are HIV-infected or AIDS-afflicted, said process comprising the steps of:
a) hybridizing an MCEF hybridization probe preparation against target nucleic acid from a person or against nucleic acid generated by amplification procedures from a person, said person being HIV-infected or AIDS-afflicted; and
b) quantifying the amount of hybridized MCEF, wherein the probe is a DNA probe molecule or an RNA equivalent of said DNA probe molecule, such that a DNA probe molecule is one that hybridizes to an MCEF DNA control molecule, said control molecule consisting of a nucleotide sequence between 14 and 70 nucleotides in length, said nucleotide sequence part of the base sequence of the cDNA insert in the plasmid deposited under ATCC accession number PTA-1502, or to a complement thereof, at a temperature that is 10° C. below the Tm calculated according to the following equation
Tm=81.5−16.6(log₁₀[Na+])+0.41(%G+C)−(600/N)
where N is the chain length in nucleotides and where the hybridization is done in aqueous solution with the Na+concentration at 1 M, and wherein the G+C content refers to the probe.

44. An assay of claim 43 wherein the target nucleic acid is RNA from a person.

45. An assay of claim 44 whercin the target nucleic acid is mRNA.

46. An immunologic assay that comprises a step wherein wherein a sample of fluid, cells or tissue, said sample preferably blood or plasma, from a patient who is HIV-infected or AIDS afflicted, is reacted with an antibody or serum reactive with a P-TEFb protein to form an antibody-protein complex, said protein being a P-TEFb protein, wherein the MCEF-type polypeptide is one that comprises at least 50 consecutive amino acids of the amino acid sequence of mammalian MCEF, said amino acid sequence being the one encoded by the MCEF cDNA insert contained in the plasmid deposited under ATCC accession number PTA-1502.
